SEQUENCE LISTING (4 sequences)

SEQ ID NO: 1 - DNA, 20 bp, Artificial Sequence
  Forward primer for actin (NM_001101)
acaatgagctgcgtgtggct

SEQ ID NO: 2 - DNA, 21 bp, Artificial Sequence
  Reverse primer for actin (NM_001101)
tctccttaatgtcacgcacga

SEQ ID NO: 3 - DNA, 21 bp, Artificial Sequence
  Forward primer for FcER1G (NM_004106)
cttttggttgaacaagcagcg

SEQ ID NO: 4 - DNA, 22 bp, Artificial Sequence
  Reverse primer for FcER1G (NM_004106)
cctttcgcacttggatcttcag

(19) United States
(12) Patent Application Publication
(10) Pub. No.: US 2005/0053950 A1
(43) Pub. Date: Mar. 10, 2005

(54) Protocol and software for multiplex real-time PCR quantification based on the different melting temperatures of amplicons

(76) Inventors: Enrique Zudaire Ubani, Columbia, MD (US); Frank Cuttitta, Adamstown, MD (US)

Correspondence Address: PERKINS COIE LLP, POST OFFICE BOX 1208, SEATTLE, WA, 98111-1208, US

(21) Appl. No.: 10/658,602

(22) Filed: Sep. 8, 2003

(51) Int. Cl. C12Q 1/68; C12P 19/34

(52) U.S. Cl. 435/6; 435/91.2

(57) ABSTRACT

The present invention provides a new protocol for quantifying multiplex real-time polymerase chain reaction (PCR). In particular, the present invention provides methods of quantifying multiple PCR products or amplicons in a single real-time PCR reaction based on the different melting temperatures (Tₘ) of each amplicon and the emission changes of double stranded DNA dyes such as SYBR Green I when amplicons are in duplex or in separation. For a specific amplicon with a Tₘ, the emission difference between the emission reading taken at a temperature below the Tₘ and the emission reading taken at a temperature above the Tₘ corresponds to the emission value of the amplicon in duplex. Accordingly, the emission difference of each amplicon in a single PCR reaction can be used to quantify each amplicon. The present invention further provides computer programs or computer products which perform the methods described herein.

### FIELD OF THE INVENTION

The present invention pertains to the field of multiplex real-time polymerase chain reaction. In particular, the invention pertains to the quantification of multiple amplicons in a single polymerase chain reaction based on the different melting temperatures of amplicons.

### BACKGROUND

Polymerase chain reaction (PCR) is a primer-directed in vitro reaction for the enzymatic amplification of a fragment of DNA. PCR involves repetitive cycles of DNA template denaturation, primer annealing to the DNA template, and primer extension. Each cycle begins with a denaturation step, during which the reaction sample is brought to a denaturing temperature and the duplex DNA template unwinds into two separated strands of DNA. In the subsequent annealing step, each oligonucleotide primer anneals or hybridizes to the complementary sequence of one separated strand of the DNA template at an annealing temperature. In the final extension step, a thermostable DNA polymerase engages in synthesizing nascent DNA by extending each primer from its 3′ hydroxyl end towards the 5′ end of the annealed DNA strand at an appropriate extension temperature. If the newly synthesized DNA strand extends to or beyond the region complementary to the other primer, it serves as a primer-annealing site and a template for extension in a subsequent PCR cycle. As a result, repetitive PCR cycles give rise to the exponential accumulation of a specific DNA fragment or amplicon whose termini are defined by the 5′ ends of the two primers. Theoretically, if the amplification efficiency is 100%, a single DNA template can produce a progeny of 2ⁿ amplicons of interest at the nth PCR cycle. The distinct ability of PCR to produce a substantive quantity of amplicons of interest from an initial nominal amount of sample DNA templates has been widely implemented in the fields of biomedical research and clinical diagnosis. For example, PCR has been used to diagnose inherited disorders and characterize forensic evidence. In particular, PCR has played a critical role in genotyping a vast number of genetic polymorphisms and identifying variations that underlie the onset of many diseases.

Multiplex PCR offers a more efficient approach to PCR, whereby multiple pairs of primers are used to simultaneously amplify multiple amplicons in a single PCR reaction. The simultaneous amplification of various amplicons decreases both the cost and turn-around time of PCR analysis, minimizes experimental variations and the risk of cross-contamination, and increases the reliability of end results. Since its inception, multiplex PCR has gained popularity in many areas of DNA testing including, gene deletion analysis, mutation and polymorphism analysis, genotyping and DNA array analysis, RNA detection, and identification of microorganisms.

However, traditional PCR and multiplex PCR are often limited to a qualitative rather than quantitative analysis of end-product amplicons. To overcome this limitation, real-time PCR has been developed to quantify amplicons during an ongoing PCR reaction. Real-time PCR is based on the principles that emission of fluorescence from dyes directly or indirectly associated with the formation of newly synthesized amplicons or the annealing of primers with DNA templates can be detected and is proportional to the amount of amplicons in each PCR cycle. The resulting emission curve can then be used to calculate the initial copy number of a nucleic acid template at the beginning of the PCR reaction. Real-time PCR eliminates the need for post PCR steps and is highly recognized for its high sensitivity, precision and reproducibility.

The simplest and cheapest real-time PCR reaction employs a double stranded DNA intercalating dye, such as SYBR Green I or ethidium bromide. The dyes emit little fluorescence of their own or in the presence of single stranded DNA and become intensely fluorescent in the presence of double stranded DNA. However, the drawback of using these dyes is that they do not recognize specific sequences or amplicons since they emit in the presence of any DNA fragment formed in a PCR reaction including undesired primer-dimer products, as long as the fragment is in duplex. This drawback may be overcome by introducing fluorescence-labeled, amplicon specific oligonucleotides or probes in real-time PCR. The fluorescence-labeled probes hybridize to an internal sequence of an amplicon and emit fluorescence after cleavage of the probe (e.g., Hydrolysis Probes) or during hybridization of one (e.g., Molecular Beacon) or two or more probes (e.g., Hybridization Probes). Most of these probes consist of a pair of dyes, a reporter dye and an acceptor dye, that are involved in fluorescence resonance energy transfer, whereby the acceptor quenches the emission of the reporter. In general, the fluorescence-labeled probes increase the specificity of amplicon quantification.

The advent of high throughput genetic testing has necessitated both qualitative and quantitative analysis of multiple genes and has led to the convergence of multiplex PCR and real-time PCR into multiplex real-time PCR. Since double stranded DNA intercalating dyes are not suitable for multiplexing due to their non-specificity, fluorescence-labeled probes have made multiplex real-time PCR possible. However, multiplex real-time PCR is limited by the availability of fluorescence dye combinations. Currently, only up to four fluorescence dyes can be detected and quantified simultaneously in real-time PCR. In addition, the cost associated with making dye-labeled probes and acquiring a PCR instrument capable of detecting multiple dye emissions simultaneously is economically unfavorable to most scientists.

Therefore, there is a need to develop methods of amplifying and quantifying multiple amplicons in a single PCR reaction for multiplex real-time PCR.

### SUMMARY OF THE INVENTION

One aspect of the invention is directed to methods for real-time monitoring and quantifying of multiple amplicons in a single multiplex real-time PCR reaction with the use of a double stranded DNA dye and the melting temperature discrepancy among the amplicons.

A double stranded DNA dye is known to fluoresce once a double stranded DNA fragment forms and fade away when the double stranded fragment unwinds into single strands or vice versa. Amplicons may be distinguished according to their unique melting temperatures (Tₘs). When a PCR reaction temperature rises above an annealing and/or extension temperature and towards a denaturing temperature, the amplicon with the lowest melting temperature denatures first, the amplicon with a higher melting temperature denatures next, and the amplicon with the highest melting temperature denatures last. The fluorescent emission of a double stranded DNA dye changes at a rate that is proportional to the rising of the reaction temperature and the incremental denaturation of amplicons. The emission difference between two emissions, one taken at a measuring temperature below the Tₘ of an amplicon when the amplicon remains double stranded and the other taken at a measuring temperature above the Tₘ when the double stranded DNA of the amplicon melts, reflects the emission amount of the amplicon in the double stranded status. The emission difference can be plotted against the number of cycles and the amount of each DNA template or amplicon may be determined in absolute or relative quantities by methods known in the art.

In one embodiment of the invention, a method of real-time monitoring and quantifying a nucleic acid template comprises the steps of: (a) thermally cycling a PCR mixture comprising a thermostable polymerase, the template nucleic acid, primers to form at least one amplicon from the template nucleic acid, and a double stranded DNA dye, (b) measuring cycle by cycle a pre-Tₘ emission of a double stranded DNA dye at a measuring temperature below a Tₘ of an amplicon and a post-Tₘ emission of the double stranded DNA dye at a measuring temperature above the Tₘ, and (c) determining an emission amount of the amplicon, which is the difference between the pre-Tₘ emission and the post-Tₘ emission. The method further comprises the step of quantifying an amount for the amplicon or the starting amount of the nucleic acid template by plotting the emission amount as a function of the number of cycles.

In another embodiment of the invention, a method for real-time monitoring and quantifying a total of n amplicons comprises the steps of: (a) determining the Tₘ of each amplicon, aligning Tₘs from low to high, wherein Tₘ₀ (T_{A} and/or T_{E}, an annealing and/or extension temperature)<Tₘ₁ (the Tₘ of the first amplicon)<Tₘ₂< . . . <Tₘ₍ₖ₋₁₎<Tₘₖ (the Tₘ of the kth amplicon)<Tₘ₍ₖ₊₁₎ . . . <Tₘₙ<Tₘ₍ₙ₊₁₎(T_{D}, the complete denaturing temperature), (b) measuring cycle by cycle a pre-Tₘ emission of a double stranded DNA dye at a measuring temperature (MT) between Tₘ₍ₖ₋₁₎ and Tₘₖ (or a pre-Tₘₖ MT) and a post-Tₘ emission of a double stranded DNA dye at a measuring temperature between Tₘₖ and Tₘ₍ₖ₊₁₎(or a post-Tₘₖ MT); and c) determining an emission amount of the kth amplicon, which is the difference between the pre-Tₘ emission and the post-Tₘ emission, wherein k is an integer and 1≦k≦n, and n is an integer and 2≦n≦35, preferably, 2≦n≦18, more preferably, 2≦n≦10, and most preferably, 2≦n≦7. The method further comprises the step of quantifying a starting amount for the kth amplicon or the nucleic acid template by plotting the emission amount of the kth amplicon as a function of the number of cycles.

In yet another embodiment of the invention, a method for real-time monitoring and quantifying a total of n amplicons comprises the steps of: (a) determining the Tₘ of each amplicon, aligning the Tₘs from low to high, wherein Tₘ₀ (T_{A} and/or T_{E})<Tₘ₁ (the Tₘ of the first amplicon)<Tₘ₂< . . . <Tₘ₍ₖ₋₁₎<Tₘₖ (the Tₘ of the kth amplicon)<Tₘ₍ₖ₊₁₎ . . . <Tₘₙ<Tₘ₍ₙ₊₁₎(T_{D}), (b) selecting measuring temperatures (MTs) between every two immediately adjacent Tₘs and aligning the measuring temperatures from low to high, wherein Tₘ₀<MT₁<Tₘ₁ (the Tₘ of the first amplicon)<MT₂<Tₘ₂< . . . <Tₘ₍ₖ₋₁₎<MTₖ<Tₘₖ (the T of the kth amplicon)<MT₍ₖ₊₁₎<Tₘ₍ₖ₊₁₎ . . . <MTₙ<Tₘₙ<MT₍ₙ₊₁₎<Tₘ₍ₙ₊₁₎, (c) measuring cycle by cycle a pre-Tₘ emission of a double stranded DNA dye at a temperature of MTₖ and a post-Tₘ emission of a double stranded DNA dye at a temperature of MT₍ₖ₊₁₎, and (d) determining an emission amount of the kth amplicon which is the difference between the pre-Tₘ emission and the post-Tₘ emission, wherein k is an integer and 1≦k≦n, and n is an integer and 2≦n≦35, preferably, 2≦n≦18, more preferably, 2≦n≦10, and most preferably, 2≦n≦7. The method further comprises the step of quantifying a starting amount for the kth amplicon or the kth nucleic acid template.

In another preferred embodiment of the invention, a method for monitoring and quantifying a first nucleic acid template and a second nucleic acid template comprises the steps of: (a) determining a first Tₘ of a first amplicon which is amplified from the first nucleic acid template and a second Tₘ of a second amplicon which is amplified from the second nucleic acid template, (b) thermally cycling a PCR mixture comprising a thermostable polymerase, the first and second template nucleic acids, primers to form the first amplicon and the second amplicon, and a double stranded DNA dye, (c) measuring cycle by cycle a first pre-Tₘ emission at a measuring temperature below the first Tₘ and a first post-Tₘ emission at a measuring temperature above the first Tₘ, (d) measuring cycle by cycle a second pre-Tₘ emission of a double strand DNA dye at a measuring temperature below the second Tₘ and a second post-Tₘ emission at the a measuring temperature above the second Tₘ, (e) determining a first emission amount which is the difference between the first pre-Tₘ emission and the first post-Tₘ emission, and (f) determining a second emission amount which is the difference between the second pre-Tₘ emission and the second post-Tₘ emission. The method further comprises the step of quantifying a starting amount of the first nucleic acid template and a starting amount of the second nucleic acid template.

In another preferred embodiment of the invention, a method for monitoring and quantifying a first nucleic acid template and a second nucleic acid template, comprising the steps of: (a) determining a first Tₘ and a second Tₘ, wherein the first Tₘ is less than the second Tₘ, (b) thermally cycling a PCR reaction comprising a thermostable polymerase, template nucleic acids, primers to form a first amplicon from the first template nucleic acid template and a second amplicon from the second nucleic acid template, and a double stranded DNA dye, (c) measuring cycle by cycle a first pre-Tₘ emission of a double stranded DNA dye at a measuring temperature between an annealing and/or extension temperature and the first Tₘ, a second pre-Tₘ emission (which is also a first post-Tₘ emission) at a measuring temperature between the first Tₘ and the second Tₘ, and (d) determining an emission amount of the first amplicon, which is the difference between the first pre-Tₘ emission and the second pre-Tₘ emission. The method further comprises the step of quantifying the amount of the first nucleic acid template based on the emission amount of the first amplicon and the amount of the second nucleic acid template based on the second emission.

In yet another preferred embodiment of the invention, the method is directed to monitoring and quantifying a first nucleic acid template with a first Tₘ and a second nucleic acid template with a second Tₘ, comprising the steps of: (a) determining the first Tₘ and the second Tₘ, wherein the first Tₘ is less than the second Tₘ, (b) thermally cycling a PCR reaction comprising a thermostable polymerase, template nucleic acids, primers to form a first amplicon from the first template nucleic acid template and a second amplicon from the second nucleic acid template, and a double stranded DNA dye, (c) measuring cycle by cycle a first emission at a measuring temperature between an annealing and/or extension temperature and the first Tₘ, a second emission at a measuring temperature between the first Tₘ and the second Tₘ, and a third emission at a measuring temperature between the second Tₘ and a total denaturing temperature, (d) determining a first emission difference which is the difference between the first emission and the second emission, and (e) determining a second emission difference which is the difference between the second emission and the third emission. The method further comprises the step of quantifying the amount of the first nucleic acid template based on the first emission difference and the amount of the second nucleic acid template based on the second emission difference.

Another aspect of the invention is directed to a computer program or software which, once stored in a computer memory and executed by a processor, performs the method comprising the step of subtracting a pre-Tₘ emission from a post-Tₘ emission or subtracting a post-Tₘ emission from a pre-Tₘ emission.

Another aspect of the invention is directed to a computer program product comprising a computer memory having a computer software stored therein, wherein the computer software when executed by a processor or in a computer performs the method comprising the step of subtracting a pre-Tₘ emission from a post-Tₘ emission or subtracting a post-Tₘ emission from a pre-Tₘ emission.

Another aspect of the invention is directed to a PCR instrument comprising a computer program product and/or a computer memory having a computer software stored therein, wherein the computer software when executed by a processor or in a computer performs the method comprising the step of subtracting a pre-Tₘ emission from a post-Tₘ emission or subtracting a post-Tₘ emission from a pre-Tₘ emission.

Other aspects of the invention and embodiments are described in the drawings, examples, and specification below.

### BRIEF DESCRIPTION OF THE DRAWINGS

FIG. 1 shows how fluorescence emission of a double stranded DNA dye is obtained at a measuring temperature (MT) in each cycle of a PCR reaction. Tₘ₁, Tₘ₂, Tₘ₍ₖ₋₁₎, Tₘₖ, Tₘ₍ₖ₊₁₎, and Tₘₙ represent the Tₘs of the 1st, 2nd, (k−1)th, kth, (k+1)th, and nth amplicons respectively. T_{A} represents an annealing temperature; T_{E} represents an extension temperature (T_{A} and T_{E} may be the same temperature); and T_{D} represents a total denaturing temperature that denatures all of the amplicons. MTₚᵣₑ₋ₖ represents a MT below Tₘₖ or a MT pre-Tₘₖ; MTₚₒₛₜ₋ₖ represents a MT above Tₘₖ or a MT post-Tₘₖ. Tₘ₍ₖ₋₁₎<MTₚᵣₑ₋ₖ<Tₘₖ<MTₚₒₛₜ₋ₖ<Tₘ₍ₖ₊₁₎. The fluorescence emission obtained at MTₚᵣₑ₋ₖ is a pre-Tₘₖ emission that corresponds to a total emission amount of duplex amplicons with Tₘs no less than Tₘₖ. The fluorescence emission obtained at MTₚₒₛₜ₋ₖ is a post-Tₘₖ emission that corresponds to a total emission amount of duplex amplicons with Tₘs higher than Tₘₖ. The difference between the pre-Tₘₖ emission and the post-Tₘₖ emission corresponds to the emission amount of the kth amplicon in duplex. k and n are integers and 1≦k≦n, and 2≦n.

FIG. 2 shows how fluorescence emission of a double stranded DNA dye is obtained at a measuring temperature (MT) between two immediately adjacent Tₘs in each cycle of a PCR reaction. Tₘ₁, Tₘ₂, Tₘ₍ₖ₋₁₎, Tₘₖ, Tₘ₍ₖ₊₁₎, and Tₘₙ represent the Tₘs of the 1st, 2nd, (k−1)th, kth, (k+1)th, and nth amplicons respectively. T_{A} represents an annealing temperature; T_{E} represents an extension temperature(T_{A} and T_{E} may be the same temperature); and T_{D} represents a total denaturing temperature that denatures all of the amplicons. MTₖ represents a MT between Tₘ₍ₖ₋₁₎and Tₘₖ; MTₖ₊₁ represents a MT between Tₘₖ and Tₘ₍ₖ₊₁₎. MTₖ can also be viewed as a MT post-Tₘ₍ₖ₋₁₎ or a MT pre-Tₘₖ. Similarly, MT₍ₖ₊₁₎can also be viewed as a MT post-Tₘₖ or a MT pre-Tₘ₍ₖ₊₁₎. Tₘ₍ₖ₋₁₎<MTₖ<Tₘₖ<MTₖ₊₁<Tₘ₍ₖ₊₁₎. The fluorescence emission obtained at MTₖ is a pre-Tₘₖ emission that corresponds to a total emission amount of duplex amplicons with Tₘs no less than Tₘₖ. The fluorescence emission obtained at MTₖ₊₁ is a post-Tₘₖ emission that corresponds to a total emission amount of duplex amplicons with Tₘs higher than Tₘₖ. The difference between the pre-Tₘₖ emission and the post-Tₘₖ emission corresponds to the emission amount of the kth amplicon in duplex. k and n are integers and 1≦k≦n, and 2≦n.

FIG. 3 shows how fluorescence emission of a double stranded DNA dye is obtained at a measuring temperature (MT) in each cycle of a PCR reaction containing at least two amplicons. Tₘ₁, and Tₘ₂, represent the Tₘs of the 1st and 2nd amplicons respectively. T_{A} represents an annealing temperature; TE represents an extension temperature (T_{A} and T_{E} may be the same temperature); and T_{D} represents a total denaturing temperature that denatures all of the amplicons. MTₚᵣₑ₋₁ represents a MT below Tₘ₁ or a MT pre-Tₘ₁; MTₚₒₛₜ₋₁ represents a MT above Tₘ₁ or a MT post-Tₘ₁. MTₚᵣₑ₋₂ represents a MT below Tₘ₂ or a MT pre-Tₘ₂; MTₚₒₛₜ₋₂ represents a MT above Tₘ₂ or a MT post-Tₘ₂. T_{A}/T_{D}<MTₚᵣₑ₋₁<Tₘ₁<MTₚₒₛₜ₋₁/MTₚᵣₑ₋₂<Tₘ₂<MTₚₒₛₜ₋₂<T_{D}. The fluorescence emission obtained at MTₚᵣₑ₋₁ is a pre-Tₘ₁ emission that corresponds to a total emission amount of both amplicons in duplex. The fluorescence emission obtained at MTₚₒₛₜ₋₁ is a post-Tₘ₁ emission that corresponds to an emission amount of the second amplicon in duplex. The difference between the pre-Tₘ₁ emission and the post-Tₘ₁ emission corresponds to the emission amount of the first amplicon in duplex. The fluorescence emission obtained at MTₚᵣₑ₋₂ is a pre-Tₘ₂ emission that corresponds to an emission amount of the second amplicons in duplex. The fluorescence emission obtained at MTₚₒₛₜ₋₂ is a post-Tₘ₂ emission that corresponds to an emission amount of background when all amplicons become single stranded. The difference between the pre-Tₘ₂ emission and the post-Tₘ₂ emission also corresponds to the emission amount of the second amplicon in duplex.

FIG. 4 shows how fluorescence emission of a double stranded DNA dye is obtained at a measuring temperature (MT) between two immediately adjacent Tₘs in each cycle of a PCR reaction containing at least two amplicons. Tₘ₁, and Tₘ₂, represent the Tₘs of the 1st and 2nd amplicons respectively. T_{A} represents an annealing temperature; T_{E} represents an extension temperature (T_{A} and T_{E} may be the same temperature); and T_{D} represents a total denaturing temperature that denatures all of the amplicons. MT₁ represents a MT between T_{A}/T_{E} and Tₘ₁; MT₂ represents a MT between Tₘ₁and Tₘ₂ (MT post-Tₘ₁ or a MT pre-Tₘ₂); and MT₃ represents a MT between Tₘ₂ and T_{D} (MT post-Tₘ₂). The fluorescence emission obtained at MT₁ is a pre-Tₘ₁ emission that corresponds to a total emission amount of both amplicons in duplex. The fluorescence emission obtained at MT₂ is a post-Tₘ₁ emission (or a pre-Tₘ₂ emission) that corresponds to an emission amount of the second amplicon in duplex. The difference between the pre-Tₘ₁ emission and the post-Tₘ₁ emission corresponds to the emission amount of the first amplicon in duplex. The fluorescence emission obtained at MT₃ is a post-Tₘ₂ emission that corresponds to a background emission when all amplicons become single stranded. The difference between the pre-Tₘ₂ emission and the post-Tₘ₂ emission corresponds to the emission amount of the second amplicon in duplex.

FIG. 5 shows how fluorescence emission of a double stranded DNA dye is obtained at a measuring temperature (MT) between two immediately adjacent Tₘs in each cycle of a PCR reaction containing at least two amplicons. FIG. 5 is similar to FIG. 4 except that MT₃ is omitted. In this situation, the difference between the pre-Tₘ₁ emission and the post-Tₘ₁ emission corresponds to the emission amount of the first amplicon in duplex. The fluorescence emission obtained at MT₂ is a post-Tₘ₁ emission (or a pre-Tₘ₂ emission) which corresponds to an emission amount of the second amplicon in duplex.

FIG. 6 shows how the emission amount of an amplicon in the presence of a double stranded DNA dye is obtained. Tₘ₁ represents the Tₘ of the amplicon. T_{A} represents an annealing temperature; T_{E} represents an extension temperature (T_{A} and T_{E} may be the same temperature); and T_{D} represents a total denaturing temperature that denatures all fragments of DNA. MTₚᵣₑ represents a MT below Tₘ₁ or a MT pre-Tₘ₁; MTₚₒₛₜ represents a MT above Tₘ₁ or a MT post-Tₘ₁. Fluorescence emission is obtained at MTₚᵣₑ (a pre-Tₘ₁ emission) and MTₚₒₛₜ (a post-Tₘ₁ emission). The difference between the pre-Tₘ₁ emission and the post-Tₘ₁ emission corresponds to the emission amount of the amplicon in duplex.

FIG. 7 shows a two-dimensional scheme that combines the use of multiple primer-based double stranded DNA dyes and multiple amplicons with various Tₘs. The first set of amplicons with Tₘs of Tₘ₁, Tₘ₂, Tₘ₍ₖ₋₁₎, Tₘₖ, Tₘ₍ₖ₊₁₎, and Tₘₙ are amplified in the presence of primer-based double stranded dye 1. The second set of amplicons with Tₘs of T′ₘ₁, T′ₘ₂, T′ₘ₍ₖ₋₁₎, T′ₘₖ, T′ₘ₍ₖ₊₁₎, and T′ₘₙ are amplified in the presence of primer-based double stranded dye II. The third set of amplicons with Tₘs of T″ₘ₁, T″ₘ₂, T″ₘ₍ₖ₋₁₎, T″ₘₖ, T″ₘ₍ₖ₊₁₎, and T″ₘₙ are amplified in the presence of primer-based double stranded dye III. The fourth set of amplicons with Tₘs of T′″ₘ₁, T′″ₘ₂, T′″ₘ₍ₖ₋₁₎, T′″ₘₖ, T′″ₘ₍ₖ₊₁₎, and T′″ₘₙ are amplified in the presence of primer-based double stranded dye IV. The xth set of amplicons with Tₘs of T^{x}ₘ₁, T^{x}ₘ₂, T^{x}ₘ₍ₖ₋₁₎, T^{x}ₘₖ, T^{x}ₘ₍ₖ₊₁₎, and T^{x}^{mn} are amplified in the presence of primer-based double stranded dye X. When these dyes emit at different wavelengths, all of these amplicons can be amplified in a single PCR reaction and measured at pertinent MTs and pertinent emission wavelengths. The emission amount of each amplicon can be obtained. Therefore, the total number of amplicons may become x*n. x, k and n are positive integers and 1 d k≦n, 1≦x and 2≦n.

FIG. 8 shows a melting curve of Amplicon I, the first negative derivative of the emission over temperature when a PCR reaction contains only Amplicon I. Amplicon I is a 125 base pair fragment of the FcER1G gene (GeneBank Accession Number NM_{—}044106) amplified from a forward sequence (SEQ ID No. 3) and a reverse sequence (SEQ ID No. 4). The peak of the curve corresponds to the Tₘ of Amplicon I which is 81.5° C.

FIG. 9 shows a melting curve of Amplicon II, the first negative derivative of the emission over temperature when a PCR reaction contains only Amplicon II. Amplicon II is a 375 base pair fragment of the Actin gene (GeneBank Accession Number NM_{—}001101) amplified from a forward sequence (SEQ ID No. 1) and a reverse sequence (SEQ ID No. 2). The peak of the curve corresponds to the Tₘ of Amplicon II which is 86.5° C.

FIG. 10 shows a melting curve of Amplicon I and II, the first negative derivative of the emission over temperature when a PCR reaction contains both amplicons. A pre-Tₘ₁ measuring temperature (MT) is set at 78° C. and a post-Tₘ₁ MT is set at 84° C.

FIG. 11 shows a 2% agarose DNA gel used to visualize PCR products. Lane A: a PCR reaction containing Amplicon I only. Lane B: a PCR reaction containing Amplicon II only. Lane (A+B): a PCR reaction containing Amplicon I and Amplicon II.

FIG. 12 shows standard and sample emission curves plotted over cycles in a PCR reaction containing only Amplicon I. The emission readings are obtained at 78° C. The dotted curves represent the emission of standard Amplicon I at serial dilutions. The solid curves represent the emission of sample Amplicon I with theoretical values of 10.5 (the left solid line) and 1.05 (the right solid line).

FIG. 13 shows the standard and sample emission curves in a PCR reaction containing only Amplicon I obtained at 84° C. Since the measuring temperature (84° C.) is 2.5° C. higher than the Tₘ of Amplicon I (81.5° C.), no emission was detected.

FIG. 14 shows the standard and sample emission curves in a PCR reaction containing only Amplicon II obtained at 78° C. The dotted curves represent the emission of standard Amplicon II at serial dilutions. The solid curves represent the emission of sample Amplicon II with theoretical values of 836 (the left solid line) and 83.6 (the right solid line).

FIG. 15 shows the standard and sample emission curves in a PCR reaction containing only Amplicon II obtained at 84° C. Since the measuring temperature (84° C.) is 2.5° C. lower than the Tₘ of Amplicon II (86.5° C.), emission readings were obtained.

FIG. 16 shows the emission curves of the standard (dotted lines) and sample (solid lines) both amplicons (Amplicon I and Amplicon II) in a single PCR reaction obtained at 78° C.

FIG. 17 shows the emission curves of the standard (dotted lines) and sample (solid lines) both amplicons (Amplicon I and Amplicon II) in a single PCR reaction obtained at 84° C.

FIG. 18 shows the emission curves of standard (dotted lines) and sample (solid lines) Amplicon I obtained by subtracting the emission as shown in FIG. 17 from the emission as shown in FIG. 16.

FIG. 19 shows the software MQ_PCR which is an Add-in for Microsoft Excel.

FIG. 20 show a dialog box displayed on a computer screen when the “Collate data” submenu is selected from the MQ_PCR. This box allows a user to open a csv file to process emission data.

FIG. 21 shows the Experiment Definition box. This function is activated from the MQ_PCR once a csv file is opened and allows a user to subtract background from emission data. Alternatively, it allow a user to subtract a post-Tₘ emission from a pre-Tₘ emission and generate the emission data or curves (FIG. 18) of the amplicon with the Tₘ. In Example VIII, the emission of Amplicon I was obtained as shown in FIG. 18.

FIG. 22 shows further analysis of the standard and sample curves of Amplicon I (FIG. 18) using a manually movable Ct line and resultant Rsquare plot (or a regression plot). The analysis results in the values of sample Amplicon I and II respectively.

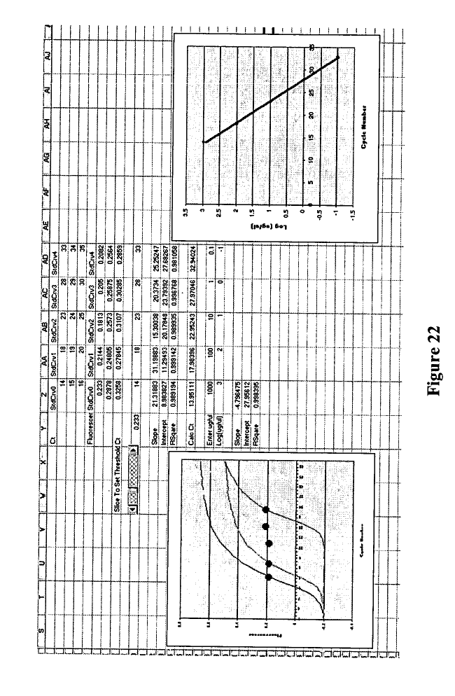

FIG. 23 show a regression line (cDNA amount vs. cycle number) obtained from the standard curves shown in FIG. 16.

FIG. 24 shows a regression line (cDNA amount vs. cycle number) obtained from the standard curves shown in FIG. 18.

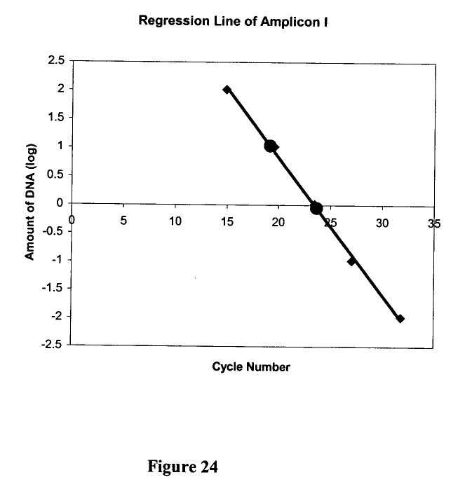

### DETAILED DESCRIPTION

One aspect of the invention is directed to methods for real-time monitoring and quantifying a plurality of nucleic acid templates in a single multiplex PCR reaction based upon the properties of at least one double stranded DNA dye and the melting temperatures of DNA fragments or amplicons which are amplified from the nucleic acid templates.

As is well known in the art, double stranded DNA dyes, such as SYBR Green™ I and ethidium bromide, are commonly used as inexpensive fluorescent dyes for real-time PCR applications. However, these dyes emit indiscriminately in the presence of double stranded nucleic acids, including PCR artifacts such as primer-dimers and spurious amplification artifacts. In addition, double stranded DNA dyes only emit one wavelength of light, making it impossible to conduct multiplex PCR with color (or wavelengths of various dyes) as a basis for discrimination. Thus, as known to the art, the nonspecific nature and lack of multiplexing ability of double stranded DNA dyes have made them undesirable for use in multiplex real-time PCR.

The melting temperature (Tₘ) of a fragment of double stranded nucleic acids is the temperature at which 50% of the fragment remains in double helix and the other 50% unwinds or separates into two single stranded complementary chains. Tₘ is affected by a number of factors, including but not limited to, salt concentration, DNA concentration, and the presence of denaturants, nucleic acid sequence, GC content, and length. Typically, each fragment of double stranded nucleic acids (e.g., amplicon) has a unique Tₘ. At a temperature below a given Tₘ at least 50% of amplicons with the Tₘ remains intact in duplex. By contrast, at a temperature above a given Tₘ, over 50% of the amplicons are expected to unwind into two single stranded nucleic acid chains.

Combining the property of double stranded DNA dyes with the unique melting temperature of each amplicon has led to unexpected advantages of using these inexpensive dyes to conduct multiplex real-time PCR according to methods described in the present invention. When a PCR reaction temperature rises from the annealing and/or extension temperature to a denaturing temperature, the amplicon with the lowest Tₘ unwinds first, the amplicon with a next higher Tₘ separates next, and the amplicon with the highest Tₘ denatures the last. Concurrently, the fluorescent emission of a double stranded DNA dye changes in proportion to the rising reaction temperature due to the incremental melting of the amplicons. The difference between two emissions, one taken at a measuring temperature below the Tₘ of an amplicon when the amplicon remains in duplex and the other taken at a measuring temperature above the Tₘ when the double stranded DNA of the amplicon unwinds, reflects the emission amount of the amplicon in duplex. The emission amount can be plotted over the number of cycles and the absolute or relative amount of the starting copy number or amount of the nucleic acid template can be determined by methods known in the art. By the same principle, it will be readily appreciated in the art that the emission amount for each amplicon in the single multiplex PCR reaction can be determined by the difference between the emission taken at a measuring temperature below a Tₘ and the emission taken at a measuring temperature above the Tₘ.

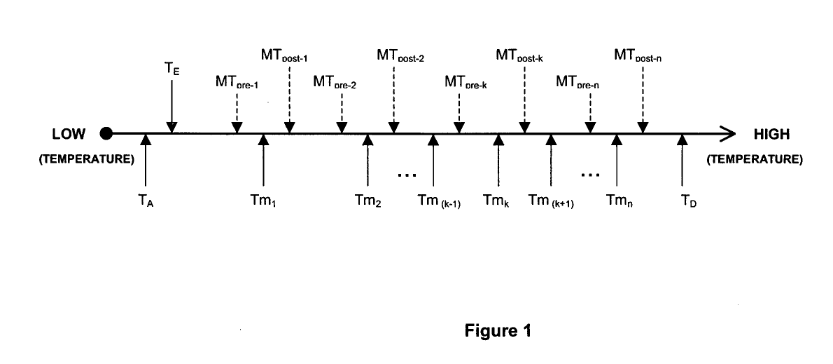

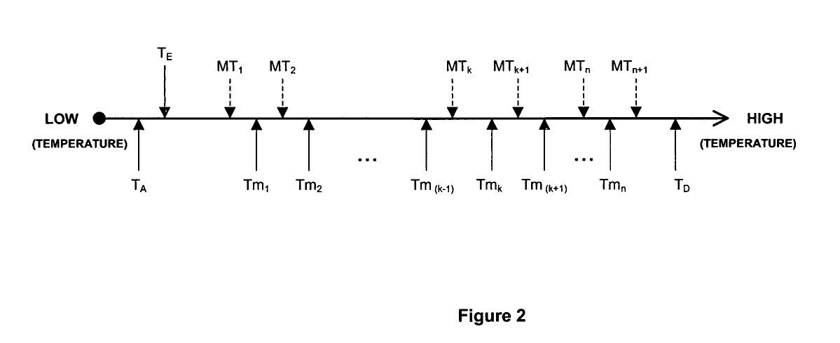

Accordingly, one aspect of the invention is directed to a method for real-time monitoring and quantifying n amplicons comprising the steps of: (a) determining the Tₘ of each amplicon, (b) aligning Tₘs from low to high, wherein Tₘ₀ (T_{A}/T_{E}, an annealing/extension temperature)<Tₘ₁ (the Tₘ of the first amplicon)<Tₘ₂< . . . <Tₘ₍ₖ₋₁₎<Tₘₖ (the Tₘ of the kth amplicon)<Tₘ₍ₖ₊₁₎ . . . <Tₘₙ<Tₘ₍ₙ₊₁₎ (T_{D}, the total denaturing temperature), (c) measuring cycle by cycle a pre-Tₘ emission of a double stranded DNA dye at a measuring temperature (MT) between Tₘ₍ₖ₋₁₎and Tₘₖ (or a pre-Tₘₖ MT) and a post-Tₘ emission of a double strand DNA dye at a measuring temperature between Tₘₖ and Tₘ₍ₖ₊₁₎(or a post-Tₘₖ MT); (d) determining an emission difference of the kth amplicon by subtracting the pre-Tₘ emission from the post-Tₘ emission (or vice versa), wherein k and n are positive integers and 1≦k≦n (See FIG. 1). The method further comprises a step of quantifying an amount for the kth through, for example, plotting the emission difference as a function of the number of cycles. In a preferred embodiment of the invention, only one emission is obtained at a measuring temperature between every two immediately adjacent Tₘs, wherein a pre-Tₘₖ MT and a post-Tₘ₍ₖ₋₁₎ MT merge into one MT (See FIG. 2).

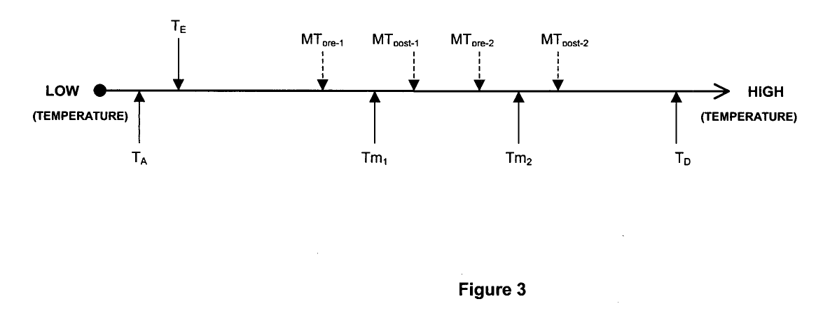

Another aspect of the present invention is directed to a method of real-time monitoring and quantifying a first nucleic acid template of a first Tₘ and a second nucleic acid template of a second Tₘ comprising the steps of (a) determining the first Tₘ and the second Tₘ; (b) thermally cycling a PCR reaction comprising a thermostable polymerase, nucleic acid templates, primers to form a first amplicon from the first nucleic acid template and a second amplicon from the second nucleic acid template, and a double stranded DNA dye; (c) measuring cycle by cycle a first pre-Tₘ emission of a double stranded DNA dye at a temperature below the first Tₘ of an amplicon and a first post-Tₘ emission of the double stranded DNA dye at a temperature above the first Tₘ; (d) measuring cycle by cycle a second pre-Tₘ emission of a double stranded DNA dye at a temperature below the second Tₘ and a second post-Tₘ emission of the double strand DNA dye at a temperature above the second Tₘ; (e) determining a first emission difference by subtracting the first pre-Tₘ emission from the first post-Tₘ emission; and (f) determining a second emission difference by subtracting the second pre-Tₘ emission from the second post-Tₘ emission (See FIG. 3). The method further comprises a step of quantifying the amount of the first nucleic acid template based on the first emission difference and the amount of the second nucleic acid template based on the second emission difference.

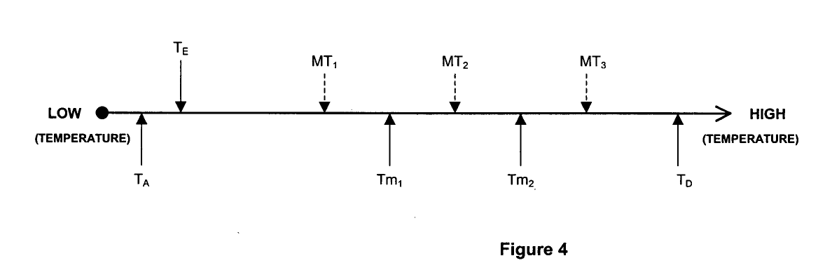

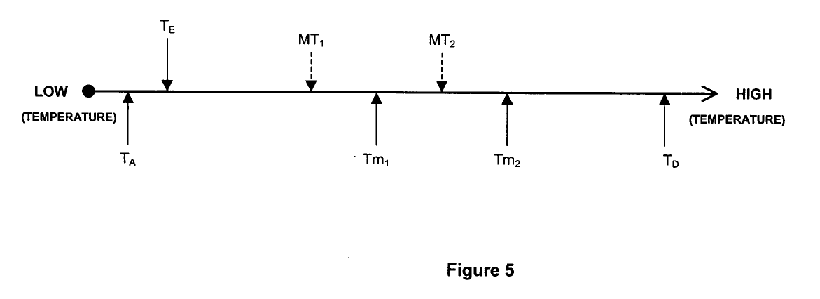

In a preferred embodiment, if the first Tₘ is less than the second Tₘ, the temperature above the first Tₘ and the temperature below the second Tₘ can be merged into one temperature which becomes a MT between the first Tₘ and the second Tₘ (See FIG. 4). In a more preferred embodiment, if the first Tₘ is less than second Tₘ, measuring the second post-Tₘ emission may be omitted, and the second post-Tₘ emission may be defined as zero (See FIG. 5).

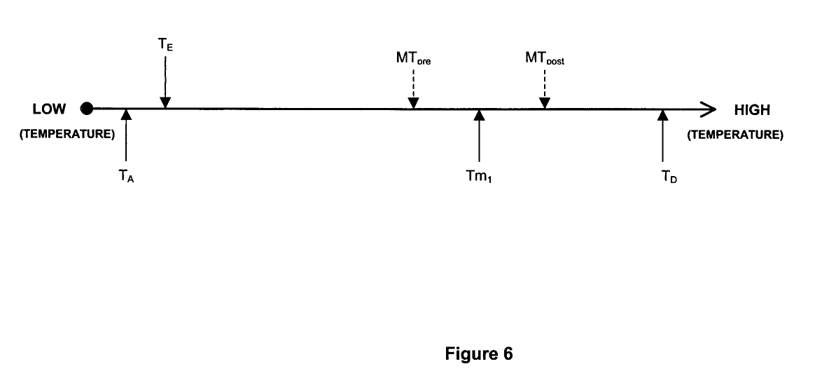

Another aspect of the present invention is directed to a method of real-time monitoring and quantifying a plurality of nucleic acid template comprises the steps of: (a) thermally cycling a PCR mixture comprising a thermostable polymerase, the template nucleic acids, primers to form at least one amplicon from the template nucleic acids, and a double stranded DNA dye, (b) measuring cycle by cycle a pre-Tₘ emission of a double stranded DNA dye at a measuring temperature below a Tₘ of an amplicon and a post-Tₘ emission of the double stranded DNA dye at a measuring temperature above the Tₘ, and (c) determining an emission amount of the amplicon which is the difference between the pre-Tₘ emission and the post-Tₘ emission (See FIG. 6). The method further comprises the steps of quantifying an amount for the amplicon or the starting amount of the nucleic acid template by plotting the emission amount as a function of the number of cycles.

DOUBLE STRANDED DNA DYES. The term “double stranded DNA dye” used herein refers to a fluorescent dye that (1) is related to a fragment of DNA or an amplicon and (2) emits at a different wavelength in the presence of an amplicon in duplex formation than in the presence of the amplicon in separation. A double stranded DNA dye can be a double stranded DNA intercalating dye or a primer-based double stranded DNA dye.

A double stranded DNA intercalating dye is not covalently linked to a primer, an amplicon or a nucleic acid template. The dye increases its emission in the presence of double stranded DNA and decreases its emission when duplex DNA unwinds. Examples include, but are not limited to, ethidium bromide, YO-PRO-1, Hoechst 33258, SYBR Gold, and SYBR Green I. Ethidium bromide is a fluorescent chemical that intercalates between base pairs in a double stranded DNA fragment and is commonly used to detect DNA following gel electrophoresis. When excited by ultraviolet light between 254 nm and 366 nm, it emits fluorescent light at 590 nm. The DNA-ethidium bromide complex produces about 50 times more fluorescence than ethidium bromide in the presence of single stranded DNA. SYBR Green I is excited at 497 nm and emits at 520 nm. The fluorescence intensity of SYBR Green I increases over 100 fold upon binding to double stranded DNA against single stranded DNA. An alternative to SYBR Green I is SYBR Gold introduced by Molecular Probes Inc. Similar to SYBR Green I, the fluorescence emission of SYBR Gold enhances in the presence of DNA in duplex and decreases when double stranded DNA unwinds. However, SYBR Gold's excitation peak is at 495 nm and the emission peak is at 537 nm. SYBR Gold reportedly appears more stable than SYBR Green I. Hoechst 33258 is a known bisbenzimide double stranded DNA dye that binds to the AT rich regions of DNA in duplex. Hoechst 33258 excites at 350 nm and emits at 450 nm. YO-PRO-1, exciting at 450 nm and emitting at 550 nm, has been reported to be a double stranded DNA specific dye. In a preferred embodiment of the present invention, the double stranded DNA dye is SYBR Green I.

A primer-based double stranded DNA dye is covalently linked to a primer and either increases or decreases fluorescence emission when amplicons form a duplex structure. Increased fluorescence emission is observed when a primer-based double stranded DNA dye is attached close to the 3′ end of a primer and the primer terminal base is either dG or dC. The dye is quenched in the proximity of terminal dC-dG and dG-dC base pairs and dequenched as a result of duplex formation of the amplicon when the dye is located internally at least 6 nucleotides away from the ends of the primer. The dequenching results in a substantial increase in fluorescence emission. Examples of these type of dyes include but are not limited to fluorescein (exciting at 488 nm and emitting at 530 nm), FAM (exciting at 494 nm and emitting at 518 nm), JOE (exciting at 527 and emitting at 548), HEX (exciting at 535 nm and emitting at 556 nm), TET (exciting at 521 nm and emitting at 536 nm), Alexa Fluor 594 (exciting at 590 nm and emitting at 615 nm), ROX (exciting at 575 nm and emitting at 602 nm), and TAMRA (exciting at 555 nm and emitting at 580 nm). In contrast, some primer-based double stranded DNA dyes decrease their emission in the presence of double stranded DNA against single stranded DNA. Examples include, but are not limited to, fluorescein (exciting at 488 nm and emitting at 530 nm), rhodamine, and BODIPY-FI (exciting at 504 nm and emitting at 513 nm). These dyes are usually covalently conjugated to a primer at the 5′ terminal dC or dG and emit less fluorescence when amplicons are in duplex. It is believed that the decrease of fluorescence upon the formation of duplex is due to the quenching of guanosine in the complementary strand in close proximity to the dye or the quenching of the terminal dC-dG base pairs.

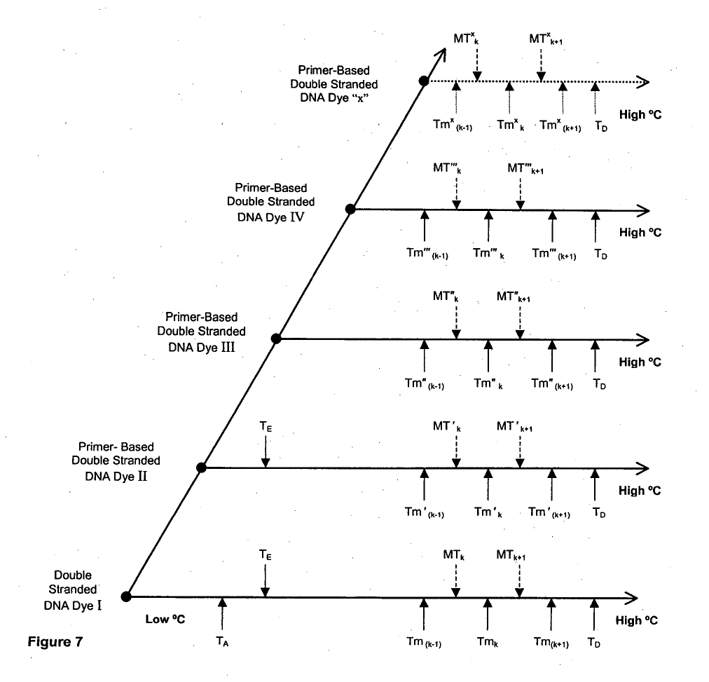

NUMBER OF AMPLICONS. The term “n” used herein refers to the total number of nucleic acid templates that can be amplified and quantified by applying the methods as described in the present invention. When only one double stranded DNA dye is added to a PCR mixture, n is an integer and 2≦n≦35, preferably, 2≦n≦18, more preferably, 2≦n≦10, even more preferably, 2≦n≦7, and most preferably, 2≦n≦5. In another preferred embodiment, “n” is 2, 3, 4, 5, 6, 7, 8, 9, or 10. If emission of various double stranded DNA dyes does not overlap, it is contemplated within the scope of this invention that more than one double stranded DNA dye can be used in a single PCR mixture. For example, a number of primer-based double stranded DNA dyes can be combined in a single PCR reaction or can be further combined with a double stranded DNA intercalating dye, as long as these dyes emit at different wavelengths. However, two double stranded DNA intercalating dyes may not be combined in a single PCR mixture. When x number of dyes are combined in a single PCR mixture, where x is an integer and x≧2, it is contemplated that the total number of nucleic acid templates in a single PCR reaction is an integer and 2≦n≦35x, preferably, 2≦n≦18x, more preferably, 2≦n≦10x, even more preferably, 2≦n≦7x, and most preferably, 2≦n≦5x (See FIG. 7).

MELTING TEMPERATURE (Tₘ). The term “melting temperature” or “Tₘ” refers to the temperature at which 50% of a given amplicon is in the double stranded conformation and 50% is in the single stranded conformation. Tₘ of any given DNA fragment or amplicon can be determined by methods well known in the art. For example, one method in the art to determine a Tₘ of a DNA fragment or an amplicon is to use a thermostatic cell in an ultraviolet spectrophotometer and measure absorbance at 268 nm as temperature slowly rises. The absorbance versus temperature is plotted, presenting an S-shape curve with two plateaus. The absorbance reading half way between the two plateaus corresponds to the Tₘ of the fragment or amplicon. Alternatively, the first negative derivative of the absorbance versus temperature is plotted, presenting a normal distribution curve. The peak of the normal curve corresponds to the Tₘ of the fragment or amplicon.

In a preferred embodiment, a calculation method commonly known as the nearest neighbor method can be used to determine the Tₘ of an amplicon. The nearest neighbor method takes into account the actual sequence of the amplicon, its length, base composition, salt concentration, entropy, and concentration. The algorithm for the nearest neighbor method is expressed as the following equation:
Tₘ=(1000ΔH)/A+ΔS+R*ln(C/4)−273.15+16.6 log[Na+]

In this equation, ΔH (Kcal.mol) represents the sum of the nearest neighbor enthalpy changes for a duplex. “A” is a constant containing corrections for helix initiation. ΔS is the sum of the nearest neighbor entropy changes. R is the Gas Constant which is 1.99 cal K⁻¹mol⁻¹. C is the concentration of the amplicon. [Na+] is the concentration of monovalent salt. The Tₘ based on the nearest neighbor method can often be calculated using software programs, which are readily available in the websites of, for example, the University of California Berkeley, Northwestern University, and Hoffman-La Roche Ltd. (e.g., www.cnr.berkeley.edu/-zimmer/oligoTMcalc.html; www.basic.nwu.edu/biotools/oligocalc.html; biochem.roche.com/fst/products.htm?/benchmate). These examples of software are well known to the art and readily available in public domain.

In another preferred embodiment, the Tₘ of an amplicon or Tₘs of multiple amplicons can be first determined by the nearest neighbor method and fine tuned or accurately determined in the presence of a double stranded DNA dye in a single PCR reaction. For example, a thermostable polymerase, nucleic acid templates for an amplicon or multiple amplicons, primers for the amplicons, a double stranded DNA dyes like SYBR Green I, and other necessary reagents are placed in a single PCR mixture. The PCR mixture is thermally cycled to amplify the amplicons for a number of cycles between a total denaturing temperature, an annealing temperature and/or an extension temperature. At the end of the PCR cycles, the mixture is heated from the annealing or extension temperature to the total denaturing temperature at a rate of 0.01° C.-3° C. per second. At the same time, the mixture is illuminated with light at a wavelength absorbed by the dye and the dye's emission is detected and recorded as an emission reading. The first negative derivative of the emission reading with respect to temperature is plotted against temperature to form a number of normal curves, and each peak of the curve corresponds to the actual Tₘ of an amplicon in the PCR reaction.

MEASURING TEMPERATURES. The term “measuring temperature” or “MT” refers to the temperature at which an emission reading of a double stranded DNA dye is taken cycle by cycle to determine the emission amount of an amplicon. When a total of n amplicons are amplified in a PCR reaction, Tₘ₀ (the annealing and/or extension temperature)<Tₘ₁(the Tₘ of the first amplicon)<Tₘ₂< . . . <Tₘ₍ₖ₋₁₎<Tₘₖ(the Tₘ of the kth amplicon)<Tₘ₍ₖ₊₁₎ . . . <Tₘₙ<Tₘ₍ₙ₊₁₎ (the total denaturing temperature), and 1≦k≦n, the kth emission amount for the kth amplicon is determined cycle by cycle by the difference between a pre-Tₘₖ emission of a double stranded DNA dye and a post-Tₘₖ emission. The pre-Tₘₖ emission is monitored and detected at a pre-Tₘₖ MT which is a measuring temperature below the Tₘₖ or between the Tₘ₍ₖ₋₁₎ and the Tₘₖ. The post-Tₘₖemission is monitored and detected at a pre-Tₘₖ MT which is a measuring temperature above the Tₘₖ or between the Tₘₖ and the Tₘ₍ₖ₊₁₎.

Alternatively, emission is measured at a measuring temperature (MT) between two immediately adjacent Tₘs, where the extension temperature is Tₘ₀ and is immediately adjacent to Tₘ₁, and the denaturing temperature is Tₘ₍ₙ₊₁₎ and is immediately adjacent to Tₘₙ.

In a preferred embodiment, “an MT below the Tₘₖ” or “an MT between Tₘ₍ₖ₋₁₎ and the Tₘₖ” refers to Tₘ₍ₖ₋₁₎<MT<Tₘₖ−0.25° C. In another preferred embodiment, the MTs are Tₘ₍ₖ₋₁₎<MT<Tₘₖ−0.5° C. In another preferred embodiment, the MT is Tₘ₍ₖ₋₁₎<MT<Tₘₖ−1.0° C. In another preferred embodiment, the MT is Tₘ₍ₖ₋₁₎<MT<Tₘₖ−1.5° C. In another preferred embodiment, the MT is Tₘ₍ₖ₋₁₎<MT<Tₘₖ−2.0° C.

In yet another preferred embodiment, “an MT above the Tₘ₍ₖ₋₁₎” or “an MT between Tₘ₍ₖ₋₁₎ and the Tₘₖ” is Tₘ₍ₖ₋₁₎+0.25° C.<MT<Tₘₖ In another preferred embodiment, the MT is Tₘ₍ₖ₋₁₎+0.5° C.<MT<Tₘₖ In another preferred embodiment, the MT is Tₘ₍ₖ₋₁₎+1.0° C.<MT<Tₘₖ In another preferred embodiment, the MT is Tₘ₍ₖ₋₁₎+1.5° C.<MT<Tₘₖ. In another preferred embodiment, the MT is Tₘ₍ₖ₋₁₎+2.0° C.<MT<Tₘₖ

In yet another preferred embodiment, “an MT between two immediately adjacent Tₘs” or “an MT between Tₘ₍ₖ₋₁₎ and the Tₘₖ” is Tₘ₍ₖ₋₁₎+0.25° C.<MT<Tₘₖ−0.25° C. In another preferred embodiment, the MT is Tₘ₍ₖ₋₁₎+0.5° C.<MT<Tₘₖ−0.5° C. In another preferred embodiment, the MT is Tₘ₍ₖ₋₁₎+1.0° C.<MT<Tₘₖ−1.0° C. In another preferred embodiment, the MT is Tₘ₍ₖ₋₁₎+1.5° C.<MT<Tₘₖ−1.5° C. In another preferred embodiment, the MT is Tₘ₍ₖ₋₁₎+2.0° C.<MT<Tₘₖ−2.0° C.

In yet another embodiment, the difference between two immediately adjacent Tₘs, for example, the difference between Tₘ₍ₖ₋₁₎ and Tₘₖ, is no less than 0.5° C., preferably no less than 1° C., more preferably no less than 2° C., even more preferably no less than 3° C., and most preferably no less than 4° C.

The term “an MTₚᵣₑ₋ₖ”, “an MT pre-Tₘₖ” or “a pre-Tₘₖ MT” used herein is interchangeable with the term “an MT below the Tₘₖ”. The term “an MTₚₒₛₜ₋ₖ”, “an MT post-Tₘₖ” or “a post-Tₘₖ MT” used herein is interchangeable with the term “an MT above the Tₘₖ”. It can be appreciated that “an MT between two immediately adjacent Tₘs” or “an MT between Tₘ₍ₖ₋₁₎ and the Tₘₖ” or “an MTₖ” or “an MT_{between}” can be viewed as “an MT above the Tₘ₍ₖ₋₁₎” and “an MT below the Tₘₖ”.

Since the first negative derivative of an amplicon's melting emission with respect to temperature is plotted to form a normal distribution curve, an ordinary person skilled in the field of statistics would readily define a MT at which a percentage of the total number of a given amplicon is in duplex or in separation. Accordingly, a measuring temperature below a Tₘ (a pre-Tₘ MT) is a temperature at which 60% of the total number of an amplicon is in duplex (double stranded form). In a preferred embodiment, a pre-Tₘ MT is a temperature at which 75% of the total number of an amplicon is in duplex. In another preferred embodiment, a pre-Tₘ MT is a temperature at which 85% of the total number of an amplicon is in duplex. In another preferred embodiment, a pre-Tₘ MT is a temperature at which 90% of the total number of an amplicon is in duplex. In another preferred embodiment, a pre-Tₘ MT is a temperature at which 95% of the total number of an amplicon is in duplex. In another preferred embodiment, a pre-Tₘ MT is a temperature at which 99% of the total number of an amplicon is in duplex.

By the same token, a measuring temperature above a Tₘ (a post-Tₘ MT) is a temperature at which 60% of the total number of an amplicon is in separation (single stranded form). In a preferred embodiment, a post-Tₘ MT is a temperature at which 75% of the total number of an amplicon is in separation. In another preferred embodiment, a post-Tₘ MT is a temperature at which 85% of the total number of an amplicon is in separation. In another preferred embodiment, a post-Tₘ MT is a temperature at which 90% of the total number of an amplicon is in separation. In another preferred embodiment, a post-Tₘ MT is a temperature at which 95% of the total number of an amplicon is in separation. In another preferred embodiment, a post-Tₘ MT is a temperature at which 99% of the total number of an amplicon is in separation.

A measuring temperature between two immediately adjacent Tₘs (an MT_{between}), for example, a first Tₘ₁ for a first amplicon and a second Tₘ₂ for a second amplicon, wherein Tₘ₁<Tₘ₂, is a temperature at which 60% of the first amplicon is in separation and 60% of the second amplicon is in duplex. In a preferred embodiment, an MT_{between} is a temperature at which 75% of the first amplicon is in separation and 75% of the second amplicon is in duplex. In another preferred embodiment, an MT_{between} is a temperature at which 85% of the first amplicon is in separation and 85% of the second amplicon is in duplex. In another preferred embodiment, an MT_{between} is a temperature at which 90% of the first amplicon is in separation and 90% of the second amplicon is in duplex. In another preferred embodiment, an MT_{between} is a temperature at which 95% of the first amplicon is in separation and 95% of the second amplicon is in duplex. In another preferred embodiment, an MT_{between} is a temperature at which 99% of the first amplicon is in separation and 99% of the second amplicon is in duplex.

EMISSION MEASUREMENT. The emission of a double stranded DNA dye is obtained, detected or recorded cycle by cycle in a PCR reaction after a PCR mixture is illuminated or excited by light with a wavelength absorbed by the dye. The term “cycle by cycle” refers to measurement in each cycle. The emission reading at a measuring temperature is taken to calculate the emission amount of an amplicon in a cycle. It is contemplated that emission can be detected, recorded, or obtained continuously or intermittently.

In a continuous recording process, the emission of the double stranded DNA dye is monitored and recorded, for example, every 50 ms, every 100 ms, every 200 ms or every 1 s, in each cycle of a PCR reaction. A three dimensional plot of time, temperature and emission can be formed. In any given cycle, the emission reading at a time point that corresponds to a desired MT is taken to determine the emission amount of the amplicon in the cycle.

In an intermittent recording process, the emission reading is taken only when the reaction temperature reaches a desired MT in each cycle. In a preferred embodiment, when a measuring temperature is reached, the PCR reaction is kept at the MT for 0.5 s to 20 s, preferably 1 s to 10 s; the emission reading is obtained, measured or recorded thereafter; and the temperature continues to rise in the PCR reaction.

The term “pre-Tₘ emission” refers to the emission reading measured, recorded or obtained at a pre-Tₘ MT. The term “post-Tₘ emission” refers to the emission reading measured, recorded or obtained at a post-Tₘ MT.

The difference between a pre-Tₘ emission and a post-Tₘ emission represents an emission amount of the amplicon with the Tₘ in a cycle. The emission amount of an amplicon reflects the change of the amplicon from duplex to separation. For example, when a pre-Tₘ emission is measured at a pre-Tₘ MT at which 99% of an amplicon is in duplex and a post-Tₘ emission is measured at a post-Tₘ MT at which 99% of the amplicon is in separation, the difference represents close to 100% of the emission of the amplicon in duplex. By the same token, when a pre-Tₘ emission is measured at a measuring temperature at which 75% of an amplicon is in duplex and a post-Tₘ emission is measured at a post-Tₘ MT at which 75% of the amplicon is in separation (25% in duplex), the difference represents close to 50% of the emission of the amplicon in duplex.

THERMAL CYCLING OF A PCR REACTION. By monitoring and measuring the emission of a double stranded DNA dye cycle by cycle, a PCR mixture is thermally cycled in a PCR instrument.

The term “thermally cycling,” “thermal cycling”, “thermal cycles” or “thermal cycle” refers to repeated cycles of temperature changes from a total denaturing temperature (T_{D}), to an annealing temperature (T_{A}), to an extension temperature (T_{E}) and back to the total denaturing temperature (T_{D}). The terms also refer to repeated cycles of a denaturing temperature (T_{D}) and an extension temperature (T_{E}), where the annealing and extension temperatures are combined into one temperature (T_{A}/T_{E}), a process known as rapid cycle PCR in the art. A total denaturing temperature (T_{D}) unwinds all double stranded amplicons into single strands. An annealing temperature (T_{A}) allows a primer to hybridize or anneal to the complementary sequence of a separated strand of a nucleic acid template or an amplicon. The extension temperature (T_{E}) allows the synthesis of a nascent DNA strand of the amplicon. Typically, T_{D} is between 92° C. and 96° C., preferably between 94° C. and 95° C. T_{A} is between 33° C. and 70° C., preferably between 45° C. and 65° C. T_{E} is between 45° C. and 80° C., preferably between 55° C. and 75° C.

The term “PCR mixture” used herein refers to a mixture of components necessary to amplify at least one amplicon from nucleic acid templates through thermal cycling. The mixture may comprise nucleotides (dNTPs), a thermostable polymerase, primers, and a plurality of nucleic acid templates. The mixture may further comprise a Tris buffer, a monovalent salt, and Mg²⁺. The PCR mixture may further comprise (1) non-acetylated bovine serum albumin to prevent chelation of the thermostable polymerase or nucleic acid templates and/or (2) glycerol as a stabilizer. The concentration of each component is well known in the art and can be further optimized by an ordinary skilled artisan.

The term “nucleic acid template” used herein refers to phosphate-deoxyribose polymer linked by phosphodiester bonds with purine and pyrimidine bases as side groups. The nucleic acid template may be double stranded or single stranded. A double stranded nucleic acid template may be obtained from DNA of virus, prokaryotes and eukaryotes, based on methods well known in the art. A single stranded nucleic acid template may be obtained from single stranded DNA (virus) or from messenger RNAs (mRNA) reverse transcribed into complementary DNA (cDNA). Reverse transcription of mRNA and the use of resulting cDNA in PCR are well known in the art.

The term “primer” used herein refers to an oligonucleotide with a length of 12 to 30 nucleotides, preferably 18 to 24 nucleotides. To amplify an amplicon from a nucleic acid template in PCR, two primers (a “forward primer” and a “reverse primer”) are designed to be complementary to two separate sequences in the nucleic acid template wherein the two sequences flank the amplicon. The length, sequence, and concentration of primers used in a PCR mixture can be determined and optimized by an ordinary skilled artisan.

When a double stranded DNA intercalating dye is used in the methods of the present invention, it is usually not necessary to label a primer with another dye. However, it is considered within the scope of the invention that a primer can be designed to contain a hairpin structure similar to a Molecular Beacon, a reporter dye, or a quencher dye, as long as the reporter dye emits at a different wavelength from the double stranded DNA intercalating dye. The amplicon amplified from the reporter dye-linked primer can be individually analyzed and quantified.

When a double stranded DNA dye is primer-based, then primers should be designed and covalently linked to the dye at a specific nucleotide or location in the primer as above mentioned.

Often one pair of primers is used to amplify one amplicon. However, it is contemplated in the present invention that one pair of primers can be used to amplify, detect and quantify more than one nucleic acid template, particularly in the case where the nucleic acid template contains mutations (alternations, one or more nucleotide substitution, deletions, or additions) in the sequence between the two primers. A well known equation used to predict changes in Tₘ (ΔTₘ) includes ΔTₘ=0.41 (% GC) if the length of two amplicons remains the same, and ΔTₘ=500/L₁−500/L₂ if the GC content is constant, wherein “% GC” refers to a percentage change of the GC content and L₁×L₂ refer to the length of a first amplicon and a second amplicon, respectively. It will be readily appreciated that mutations occurring between the pair of primers in the nucleic acid templates will be reflected in the difference in Tₘs that can be detected and quantified within the scope of the present invention. It is considered within the scope of the present invention that one pair of primers in the present invention can be used to discover unknown mutations in the sequence of nucleic acid templates flanked by the primers, since the amplicon with a mutated sequence may reveal a Tₘ different from that of the wild-type sequence.

In a preferred embodiment of the present invention, the ability of one pair of primers to detect and quantify more than one amplicon with different Tₘs is useful in identifying and quantifying highly variable regions of a nucleic acid template subject to frequent mutation. It is also useful in detecting and quantifying nucleic acid templates with alternative gene splicing occurring in a region between the primers. Moreover, it is useful in detecting and quantifying single nucleotide polymorphisms (SNPs) in nucleic acid templates. SNPs comprise the most abundant category of DNA sequence variation, occurring at a rate of about 1 per 500 nucleotides in coding sequences and at a higher rate in non-coding sequences. SNPs are amenable for high-throughput genotyping with, for example, DNA arrays and mass spectrometry. The difference in Tₘ (ΔTₘ) between a homoduplex (two single strands that are in perfect match) and a heterodulex (two single strands that are not in perfect match) amplicon of 100-150 base pairs differing by only a single nucleotide substitution is reportedly 1-5° C. By the same token, the difference in Tₘ among the homoduplex of a wild-type amplicon, the homoduplex of a mutant amplicon, and the heteroduplex of the two amplicons can be distinguished and utilized for quantifying the three amplicons according to the methods provided in the present invention.

The term “amplicon” refers to a fragment of DNA amplified from a thermostable polymerase using a pair of primers (a forward primer and a reverse primer) in PCR. As mentioned, a pair of primers may produce more than one fragment of DNA if the nucleic acid templates contain mutant and wild-type sequences. Each amplicon has its specific sequence, length, and Tₘ. In a preferred embodiment, the length of the amplicon is from 50 base pairs to 1000 base pairs, more preferably from 80 base pairs to 500 base pairs. It is contemplated that primer pairs can be designed according to methods known in the art so that amplicons flanked by primer pairs have different Tₘs.

It is contemplated that a PCR mixture of the present invention may further include one or more fluorescence resonance energy transfer (FRET) based probes. FRET based probes are well known in the art and include, for example, Taqman probes, double-dye oligonucleotide probes, Eclipse probes, Molecular Beacon probes, Scorpion probes, Hybridization probes, ResonSense probes, Light-up probes, Hy-Beacon probes. A FRET probe may be used to specifically analyze one or more amplicons among a plurality of amplicons, distinguish two amplicons with substantially the same Tₘ, further increase the number of amplicons in a single PCR reaction, and analyze and quantify a plurality of amplicons in a two dimensional multiplex system comprising multiple wavelength emission and multiple Tₘs. When a FRET based probe is used, an amplicon may further comprise a reporter dye covalently linked to the amplicon through the probe wherein the reporter dye is not a double stranded DNA dye. It is also contemplated that an amplicon may comprise a peptide nucleic acid to which a FRET based dye is tethered.

Thermal-cycling of a PCR mixture is performed in a PCR instrument. PCR instruments that may be used herein include the Smart Cycler System, the Idaho Rapid Cycler, the Carbett Roter-Gene System, the GeneAmp 5700 Sequence Detection System, the ABI Prism7000, 7700 & 7900 Sequence Detection Systems, the iCycler System, the MX-4000 Multiplex Quantitative PCR System, the DNA Engine Opticon System, and MJ Research's DNA Engine Opticon System.

Quantification of Amplicons or Nucleic Acid Templates.

As a PCR mixture undergoes thermal cycling, the emission amount of an amplicon (the difference between a pre-Tₘ and a post-Tₘ emission readings for the amplicon) is recorded and plotted over the number of cycles to form an emission versus cycle plot. In the initial cycles, there is little change in the emission amount that appears to be a baseline or a plateau in the plot. As thermal cycling continues, an increase in emission amount above the baseline may be expected to be observed, which indicates that the amplified amplicon has accumulated to the extent that fluorescence emission of a double stranded dye in the presence of the amplicon exceeds the detection threshold of a PCR instrument. An exponential increase in emission amount initiates the exponential phase and eventually reaches another plateau when one of the components in the PCR mixture becomes limiting. The plotting usually produces an S-shape curve with two plateaus at both ends and an exponential phase in the middle. In the exponential phase, the emission amount of the amplicon is increasing by (1+E) fold over the previous amount of each cycle, wherein E is the efficiency of amplification, which ideally should be 100% or 1. It is commonly known that the higher the starting amount of the nucleic acid template from which an amplicon is amplified, the earlier an increase over baseline is observed. As is well known in the art, the emission versus cycle plot provides significant information for attaining the initial copy number or amount of the nucleic acid template.

As known in the field of real-time PCR, the unknown amount of a nucleic acid template is quantified by comparing the emission versus cycle plot of the template (or the amplicon) with standardized plots. The standard plots are formed when a known nucleic acid template is purified, quantitated and then diluted into several orders of magnitude (for example, 10⁰, 10⁻¹, 10⁻², 10⁻³, 10⁻⁴, 10⁻⁵). Each dilution of the template is placed into a separate PCR mixture for thermal cycling. The emission amount of each dilution is plotted onto the same graph which shows a multiple of S-shaped curves (“standard plots”) with the exponential phase of the highest starting amount of the template occurring the earliest in thermal cycling and the lowest amount appearing the last. A fix emission line or a cycle threshold line can be set horizontally above the baseline of the S-curves to intersect with the S-shaped curves. The threshold cycle (Cₜ) is the value of the x-axis (the number of cycles) at which the cycle threshold line intersects one of the S-shaped curves. The logarithm of each initial diluted amount for the set of standard plots is plotted with respect to its corresponding C_{T}, forming a near perfect straight line. This line is a regression line with a regression square (R Square) substantially close to 1. To calculate the Cₜ of the sample template of interest, the sample template emission versus cycle plot is superimposed upon the standard plots and the C_{T} Of the template is obtained where the fix emission line intersects. The C_{T} of the template is then compared to the regression line and the starting copy number or amount of the template is obtained.

In one embodiment of the present invention, when a plurality of nucleic acid templates are amplified to form a plurality of amplicons, each amplicon is preferably compared with a standard curve formed by the same amplicon. The amplicon that is used to form a standard curve can be obtained through PCR or can be synthesized. A single amplicon per dilution per PCR mixture can be used to form the standard curve. Preferably, at each dilution, a plurality of amplicons are placed in a single PCR mixture and emission readings of each amplicon can be measured and plotted to form a standard curve based on methods described in the present invention.

The starting amount of a nucleic acid template in a sample can also be determined by normalizing the template to a house keeper gene or a normalizer in relative relationship to a calibrator without using a standard curve. For example, GAPHD (glyceraldehyde 3-phosphate dehydrogenase) and β-actin are commonly regarded a suitable house keeper nucleic acid templates or normalizer templates due to their abundance and constant levels of expression. The calibrator can be an untreated sample or a specific cell, tissue or organ used for the normalization of treated samples or targeted cells. If the efficiency of the amplification for both an amplicon and the normalizer is presumed to be 1, then the relative starting amount of the nucleic acid template (the amplicon), normalized to the normalizer and relative to the calibrator, equals to 2^{-ΔΔCT}, wherein ΔΔC_{T}=ΔC_{T} of the sample −ΔC_{T} of the calibrator and ΔC_{T}=the normalizer C_{T}−the nucleic acid template C_{T}.

Often, the amplification efficiency of each amplicon differs. The efficiency for an amplicon in a PCR reaction can be determined from the following efficiency equation:
E=(Emission_{A}/Emission_{B})^{1/(CT,A-CT,B)}−1

Emission_{A} and Emission_{B} are two emission readings taken at point A and B in the exponential phase of the S-shape curve of the amplicon. C_{T, A} and C_{T, B} are corresponding C_{T}S of points A and B. If follows that the relative amount of the nucleic acid template when normalized to the normalizer, relative to the calibrator, and corrected by amplification efficiency, equals to:
E_{Template}^{ΔCT(Template)}/E_{Normalizer}^{ΔCT(Normalizer)} wherein ΔCT=calibrator C_{T}−template C_{T}. E_{Template} refers to the amplification efficiency of the nucleic acid template (its corresponding amplicon). E_{Normalizer} refers to the amplification efficiency of a normalizer.

Other algorithms commonly used to quantify the amount of an amplicon can be found in www.wzw.tum.de/qene-quantification/index.shtml.

In one embodiment of the present invention, a plurality of nucleic acid templates of interest are amplified and quantified in a single PCR mixture. The starting amount of each nucleic acid template can be simultaneously calculated and normalized to a normalizer. It is also contemplated that a plurality of nucleic acid templates and a normalizer template can be monitored and amplified in the same PCR reaction. It is further contemplated that more than one housekeeper template or normalizer can be amplified along with multiple nucleic acid templates in a single PCR reaction. It is further contemplated that the relative amount among these templates or the ratios between or among these templates can be determined from a single PCR mixture.

In a preferred embodiment of the present invention, a method or software for expediting and optimizing the formation of a standard curve comprises: (a) a computer program code for forming a movable scroll bar in Microsoft Excel, (b) a computer program code for determining threshold cycle (CT) number when the scroll bar is manually placed across curves in an emission over cycle plot, and (c) a computer program code for translating the threshold cycle number and the logarithm of initial amounts of nucleic acids template into a regression curve.

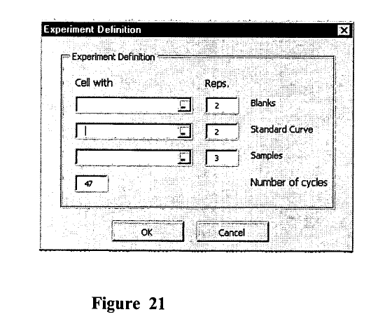

The scroll bar developed herein refers to a cycle threshold line as mentioned earlier, which is set above the baseline of S-curves in a standard plot. In one embodiment of the present invention, the scroll bar can be moved up and down in the exponential phase of S-shape curves of plots and each C_{T} value intersected with the scroll bar is detected and automatically recorded. In the meantime, the logarithm of the known amount versus C_{T} value is automatically plotted as a standard curve, and RSquare is automatically calculated. Simultaneously, the C_{T}S of one or more amplicons with unknown amount is determined when the scroll bar passes and each amplicon's amount is calculated automatically from the standard curve. It can be readily appreciated that this method or software easily allows a user to select the best possible standard curve with the highest possible RSquare at a fingertip and save the user a significant amount of time. For an example of this method, see FIG. 21.

COMPUTER PROGRAM AND/OR PRODUCT. Generally, the difference between a pre-Tₘ emission and a post-Tₘ emission can be calculated manually by subtracting a pre-Tₘ emission from a post-Tₘ emission, or vice versa, once the emission values are acquired through a PCR instrument. However, it is frequently desirable to automate the calculation through the use of a computer system.

A computer system according to the present invention refers to a computer or a computer readable medium designed and configured to perform some or all of the methods as described herein. A computer used herein may be any of a variety of types of general-purpose computers such as a personal computer, network server, workstation, or other computer platform now or later developed. As commonly known in the art, a computer typically contains some or all the following components, for example, a processor, an operating system, a computer memory, an input device, and an output device. A computer may further contain other components such as a cache memory, a data backup unit, and many other devices. It will be understood by those skilled in the relevant art that there are many possible configurations of the components of a computer.

A processor used herein may include one or more microprocessor(s), field programmable logic arrays(s), or one or more application specific integrated circuit(s). Illustrative processors include, but are not limited to, Intel Corp's Pentium series processors, Sun Microsystems' SPARC processors, Motorola Corp.'s PowerPC processors, MIPS Technologies Inc.'s MIPs processors, and Xilinx Inc.'s Vertex series of field programmable logic arrays, and other processors that are or will become available.

A operating system used herein comprises machine code that, once executed by a processor, coordinates and executes functions of other components in a computer and facilitates a processor to execute the functions of various computer programs that may be written in a variety of programming languages. In addition to managing data flow among other components in a computer, an operating system also provides scheduling, input-output control, file and data management, memory management, and communication control and related services, all in accordance with known techniques. Exemplary operating systems include, for example, a Windows operating system from the Microsoft Corporation, a Unix or Linux-type operating system available from many vendors, any other known or future operating systems, and some combination thereof.

A computer memory used herein may be any of a variety of known or future memory storage devices. Examples include any commonly available random access memory (RAM), magnetic medium such as a resident hard disk or tape, an optical medium such as a read and write compact disc, or other memory storage devices. A memory storage device may be any of a variety of known or future devices, including a compact disk drive, a tape drive, a removable hard disk drive, or a diskette drive. Such types of memory storage device typically read from, and/or write to, a computer program storage medium such as, respectively, a compact disk, magnetic tape, removable hard disk, or floppy diskette. Any of these computer program storage media, or others now in use or that may later be developed, may be considered a computer program product. As will be appreciated, these computer program products typically store a computer software program and/or data. Computer software programs, also called computer control logic, typically are stored in system memory 120 and/or the program storage device used in conjunction with memory storage device 125.

In one embodiment, a computer program product is described comprising a computer memory having a computer software program stored therein, wherein the computer software program when executed by a processor or in a computer performs methods according to the present invention. In a preferred embodiment, a computer program product comprises a computer memory having a computer software program stored therein, wherein the computer software program performs a method comprising the step of taking the difference between a pre-Tₘ emission and a post-Tₘ-emission.

An input device used herein may include any of a variety of known devices for accepting and processing information from a user, whether a human or a machine, whether local or remote. Such input devices include, for example, modem cards, network interface cards, sound cards, keyboards, or other types of controllers for any of a variety of known input function. An output device may include controllers for any of a variety of known devices for presenting information to a user, whether a human or a machine, whether local or remote. Such output devices include, for example, modem cards, network interface cards, sound cards, display devices (for example, monitors or printers), or other types of controllers for any of a variety of known output function. If a display device provides visual information, this information typically may be logically and/or physically organized as an array of picture elements, sometimes referred to as pixels.

As will be evident to those skilled in the relevant art, a computer software program of the present invention can be executed by being loaded into a system memory and/or a memory storage device through one of the above input devices. On the other hand, all or portions of the software program may also reside in a read-only memory or similar type of memory storage device, such devices not requiring that the software program first be loaded through input devices. It will be understood by those skilled in the relevant art that the software program or portions of it may be loaded by a processor in a known manner into a system memory or a cache memory or both, as advantageous for execution.

As will be appreciated by those skilled in the art, a computer program product of the present invention, or a computer software program of the present invention, may be stored on and/or executed in a PCR instrument and used to calculate the amount of each amplicon. For example, a computer software of the present invention can be installed in, for example, the Smart Cycler System, the Idaho Rapid Cycler, the Carbett Roter-Gene System, the GeneAmp 5700 Sequence Detection System, the ABI Prism7000, 7700 & 7900 Sequence Detection Systems, the iCycler System, the MX-4000 Multiplex Quantitative PCR System, the DNA Engine Opticon System, and MJ Research's DNA Engine Opticon System.

However, it is not necessary that the computer program product or the computer software program be stored on and/or executed in a PCR instrument. Rather, the computer product or software may be stored in a separate computer or a computer server that connects to the PCR instrument through a data cable, a wireless connection, or a network system. As commonly known in the art, network systems comprise hardware and software to electronically communicate among computers or devices. Examples of network systems may include arrangement over any media including Internet, Ethernet 10/1000, IEEE 802.11x, IEEE 1394, xDSL, Bluetooth, 3G, or any other ANSI approved standard. When the computer is linked to a PCR instrument through a network system, the emission data are sent out through an output device of the PCR instrument and received through an input device of a computer having the computer program product or software. The computer program product or the software then processes the data and calculates the emission amount of an amplicon in each cycle and presents resulting data (e.g., an emission amount in a file, an emission over cycle plot, the amount of each amplicon, and/or a Rsquare value) through an output device associated with the computer. It is also contemplated that the emission data can be stored in a server in a network system, the computer software of the present invention is executed in the server or through a separate computer, and resulting information is presented to a user in the presence of an output of the computer.

APPLICATIONS IN MICROARRAY. Microarray technology allows a large number of molecules or materials to be synthesized or deposited in the form of a matrix on a supporting plate or membrane, commonly known as a chip. In a preferred embodiment, microarray technology allows a large number of molecules (also known as probe molecules) to be synthesized or deposited on a single chip and to interact with unknown molecules (target molecules) to obtain the information about the nature, identity, or quantity of the target molecules. The interaction between probe molecules and target molecules is preferably hybridization, and more preferably base pairing hybridization. Illustrative examples of microarray include biochip, DNA chip, DNA microarray, gene array, gene chip, genome chip, protein chip, microfluidics based chip, combinatory chemical chip, combinatory material based chip.

In a preferred embodiment, microarray is an oligonucleotide array or a spotted cDNA array. In the oligonucleotide array, an array of oligonucleotides (20-80-mer oligonucleotide, preferably 30-mer) or peptide nucleic acid probes are synthesized either in situ (on-chip) or by conventional synthesis followed by on-chip immobilization. The oligonucleotide array is then exposed to labeled target DNA molecules, hybridized, and the identity and/or abundance of complementary sequences are determined. In the spotted cDNA array, probe cDNAs (200 bp to 5000 bp long) are immobilized onto a solid surface such as microscope slides using robotic spotting. The spotted cDNA array is then exposed or hybridized with different fluorescently labeled target molecules derived from RNA of various samples of interest. As known in the art, oligonucleotide arrays can be used for applications including identification of gene sequence/mutations and single nucleotide polymorphisms and monitoring of global gene expression. The spotted cDNA arrays can be used for, for example, the studying of the genome-wide profile or a pattern of mRNA expression.

Microarray data reflect the interaction between probe molecules and target molecules. As commonly known in the art, an illustrative example of microarray data refers to fluorescence emission readings derived from microarray when target molecules are labeled with a set of fluorescent dyes (for example, Cy3 and Cy5). The labeled target molecules interact or hybridize with the probe molecules synthesized or deposited on the mircoarray and the emission reading of fluorescence is detected through any means known in the art. The emission in the microarray is scanned and collected to produce a microarray image. Emission in each array cell in the microarray is taken to collectively produce microarray data.

It is contemplated that the emission of a double stranded DNA dye in the presence of double stranded hybridization between probe and target molecules can be used in microarray. For example, microarray plates can be treated with a double stranded DNA dye and emission of the dye can be detected continuously or discontinuously over rising temperature from an annealing temperature to a total denaturing temperature. In the cDNA spotted array, SNPs or gene splicing can be detected in each array cell when the emission unexpectedly drops or rises in comparison with wild type genes or fragments.

ADVANTAGES. From the foregoing description, it will be readily appreciated that methods provided in the present invention attain significant advantages not heretofore present in the art. For example, the methods in the present invention substantially reduce the cost and time of performing multiplex real-time PCR. Although other fluorescence dyes may be co-employed in a PCR reaction, one double stranded DNA dye, such as SYBR Green I, is sufficient to quantify a plurality of amplicons. It becomes unnecessary to incur the expense of labeling one or two dyes on probes or acquiring a PCR instrument suitable for simultaneously distinguishing emission at various wavelengths.

For another example, the methods in the present invention substantially increase the specificity of amplicons even in the presence of a non-specific double stranded DNA dye, since the specificity in the present invention emanates directly from the inherent properties of the amplicons, which are their unique melting temperatures. However, the specificity of methods currently known in the art is determined indirectly from the specificity of primers, probes, or dye emission wavelengths in relation to amplicons.

For another example, the methods in the present invention substantially increase the number of amplicons to be amplified and quantitated in a single multiplex real-time PCR reaction. As known in the art, real-time quantification in multiplex PCR depends on the availability of fluorescence dyes and the discrimination of their emission wavelength. The overlap of emission interferes with the emission readings of dyes. Accordingly, so far only up to four dyes can be used for simultaneous quantification. The methods in the present invention eliminate the need for multiple dyes, since quantification depends on the melting temperature of each amplicon and the difference between a pre-Tₘ emission and a post-Tₘ emission emitted from a single double stranded DNA dye. The number of amplicons in the present invention depends on the number of Tₘs among the amplicon spanning from an annealing/extension temperature and a denaturation temperature and a PCR instrument's limit on the separation and detection of the emission difference. In addition, the number of amplicons can be further multiplied when one or more double stranded dyes are combined and/or when fluorescence labeled probes are combined.

For another example, the methods in the present invention obviate the need to design multiple primers for single nucleotide polymorphism or any mutations occurring in an amplicon. Any mutation, whether it is a single base substitution and/or deletion and/or addition, an oligonucleotide substitution and/or deletion and/or addition, or an alternative splice product, can be detected and quantified in a single reaction, as long as the mutant amplicon has a different melting temperature from the wild type amplicon.

### REFERENCES

Papers from Scientific Journals
Ball, T. et al, Improved mRNA Quantification in LightCycler RT-PCR, Int. Arch Allergy Immunol. 130: 82-86 (2003).
Bohling, S. D. et al., Rapid Simultaneous Amplification and Detection of the MBR/JH Chromosomal Translocation by Fluorescence Melting Curve Analysis, Am. J. Path. 154: 97-103 (1999).
Brownie et al., The Elimination of Primer-Dimer Accumulation in PCR, Nucleic Acids Res. 25: 3235-3241 (1997).
Bustin, S. Absolute quantification of mRNA using real-time reverse transcription polymerase chain reaction assays, J. Mol. Endocrinol. 25: 169-193 (2000).
Bustin, S. Quantification of mRNA using Real-Time Reverse Transcription PCR (RT-PCR): Trends and Problems, J. Mol. Endocrinol. 29: 23-39 (2002).
Caplin, B. E. et al., LightCycler™ Hybridization Probes—The most direct way to monitor PCR amplification and mutation detection, Biochemica 1: 5-8 (1999).
Cha & Thilly, Specificity, Efficiency, and fidelity of PCR, PCR Methods. Appl. 3: S18-S19 (1993).
Chamberlian et al, Deletion Screening of the Duchenne Muscular Dystrophy Locus via Multiplex DNA Amplification, Nucleic Acids Res. 16: 11141-56 (1988).
Donohoe, G. et al., Rapid Single-Tube Screening of the C282Y Hemochromatosis Mutation by Real-Time Multiplex Allele-specific PCR without Fluorescent Probes, Clin. Chem. 46:1540-1547 (2000).
Edwards & Gibbs, Multiplex PCR: Advantages, Developments and Applications, PCR Meth. Appl. 3: S65-75 (1994).
Elnifro et a!, Multiplex PCR: Optimization and Application in Diagnostic Virology, Clin. Microbiol. Rev. 13: 559-570 (2000).
Erlich et a!, Recent Advances in the Polymerase Chain Reaction, Science 252: 1643-51 (1991)
Freeman, W. M. et al, Quantitative RT-PCR: Pitfalls and Potential, Biotechniques 26: 112-125 (1999).
French, D. et al, HyBeacon probes: a new tool for DNA sequence detection and allele discrimination, Mol. Cell Probes 15: 363-74 (2001).
Ginzinger, D., Gene Quantification Using Real-Time Quantitative PCR: An Emerging Technology Hits the Mainstream, Exp. Hematol.30: 503-512 (2002)
Giulietti, A. et al., An Overview of Real-Time Quantitative PCR: Applications to Quantify Cytokine Gene Expression, Methods 25: 386-401 (2001).
Halford, W. P., The essential prerequisites for quantitative RT-PCR, Nature Biotechnol. 17: 835 (1999).
Heid, C. A. et al., Real-time quantitative PCR, Genome Res. 6: 986-84 (1996).
Henegariu, O. et al., Multiplex PCR: Critical Parameters and Step-by-Step Protocol, Biotechniques 23: 504-511 (1997).
Holland, P. et al., Detection of specific polymerase chain reaction product by utilizing the 5′-3′ exonuclease activity of thermus aquaticus, Proc. Natl. Acad, Sci. USA 88: 7279-7280 (1991).
Howell, W. et al., iFRET: an improved fluorescence system for DNA-melting analysis, Genome Res. 12:1401-7 (2002).
Ju, J. et al, Fluorescence energy transfer dye-labeled primers for DNA sequencing and analysis, Proc. Natl. Acad. Sc. USA 92: 4347-4351 (1995).
Kampke, T. et al., Efficient Primer Design Algorithms, Bioinformatics 17: 214-225 (2001).
Klein, D., Quantification using real-time PCR technology: applications and limitations, Trends in Mol. Med. 8: 257-260 (2002).
Kreuzer, K A et a!, LightCycler Technology for the Quantification of bcr/abl Fusion Transcripts, Cancer Res. 59: 3171-3174 (1999).
Kutyavin, I. V. et al, 3′-minor groove binder-DNA probes increase sequence specificity at PCR extension temperatures, Nucleic Acids Res. 28: 655-61 (2000).
Landt, O. et a!, LightCycler™ Technical Note. Selection of Hybridization Probe Sequences for Use with the LightCycler™. www.TIB-MOLBIOL.de
Li & Hood, Multiplex Genotype Determination at a DNA Sequence Polymorphism Cluster in The Human Immunoglobulin Heavy-Chain Region, Genomics 26: 199-206 (1995).
Lin et al, Multiplex Genotype Determination at a Large Number of Gene Loci, Proc. Natl. Acad. Sci. USA 93: 2582-2587 (1996).
Lipsky, R. H. et al., DNA Melting Analysis for Detection of Single Nucleotide Polymorphisms, Clin. Chem. 47: 635-644 (2001).
Liu, W. et al, A New Quantitative Method of Real-time Reverse Transcription Polymerase Chain Reaction Assay Based on Simulation of Polymerase Chain Reaction Kinetics, Anal. Biochem. 302: 52-59 (2002).
Liu, W. et al, Validation of a Quantitative Method for Real-time PCR Kinetics, Biochem. Biophy. Res. Commun. 294: 347-353 (2002).
Livak, K. J. et al, Analysis of Relative Gene Expression Data Using Real-Time Quantitative PCR and the 2-^{ΔΔC}T Method, Methods 25: 402-408 (2001).
Mackay, I. M. et al, Real-Time PCR in Virology, Nucleic Acids Res. 30: 1292-1305 (2002).
Marie, D. et al, Application of the Novel Nucleic Acid Dyes YOYO-1, YO-PRO-1, and PicoGreen for Flow Cytometric Analysis of Marine Prokaryotes, Applied Environ. Microbio. 62:1649-1655 (1996)
Markoulatos et al, Multiplex Polymerase Chain Reaction: A Practical Approach, J. Clin. Lab. Anal. 16: 47-51 (2002).
Molenaar, C. et al, Linear 2′ O-Methyl RNA probes for the visualization of RNA in living cells, Nucleic Acids Res. 29: E89-9 (2001)
Mullis, K. et al, in Methods in Enzymology 155: 335 (1987).
Nazarenko, I. et al, Effect of Primary and Secondary Structure of Oligodeoxyribonucleotides on the Fluorescent Properties of Conjugated Dyes, Nucleic Acids Res. 30: 2089-2195 (2002).
Pfaffl, M., A New Mathematical Model for Relative Quantification in Real-Time RT-PCR, Nucleic Acids Res. 29: 2002-2007 (2001).
Pfaffl, M., Development and Validation of an Externally Standardized Quantitative Insulin-like Growth Factor-1 RT-PCR Using LightCycler SYBR Green I Technology, Biochemica. 2: 13-16 (2000).
Pfaffl, M. et al, Validities of mRNA Quantification Using Recombinant RNA and Recombinant DNA External Calibration Curves in Real-Time RT-PCR, Biotechnol. Let. 23: 275-282 (2001).
Rapid Cycle Real-Time PCR (Meuer, S., Wittwer C., and Nakagawara K., Eds.)(2001).
Raja, S. et al, Temperature-controlled Primer Limit for Multiplexing of Rapid, Quantitative Reverse Transcription-PCR Assays: Application to Intraoperative Cancer Diagnostics, Clinical Chemistry 38: 1329-1337 (2002).
Ramakers, C. et al, Assumption-free analysis of quantitative real-time polymerase chain reaction (PCR) data, Neuroscience Let. 339: 62-66 (2003).
Riccelli, P. et al, DNA Sequence Context and Multiplex Hybridization Reactions: Melting Studies of Heteromorphic Duplex DNA Complexes, J. Am. Chem. Soc. p. 141-50 (2003).
Ririe, Kirk M. et al, Product Differentiation by Analysis of DNA Melting Curves during the Polymerase Chain Reaction, Anal. Biochem. 125: 154-160 (1997).
Rithidech et al, Combining Multiplex and Touch Down PCR to Screen Murine Microsatellite Polymorphism, Bio. Techniques 23: 36-45 (1997).
Roberston & Walsh-Weller, An Introduction to PCR Primer Design and Optimization of Amplification Reactions, Meth. Mol. Biol. 98: 121-154 (1998).
Roux, Optimization and Troubleshooting in PCR, PCR Meth. Appl. 4: S185-S194 (1995).
Saiki, Enzymatic Amplification of β-Actin Genomic Sequences and Restriction Site Analysis for Diagnosis of Sickle Cell Anemia, Science 230: 1350-54 (1985).
Schmittgen, T. D. et al., Real-Time Quantitative PCR, Methods. 383-385 (2001).
Schmittgen, T. D. et al, Quantitative Reverse Transcription-Polymerase Chain Reaction to Study mRNA Decay: Comparison of Endpoint and Real-Time Methods, Anal. Biochem. 285:194-204 (2000).
Shi, Enabling Large-Scale Pharmacogenetic Studies by High-throughput Mutation Detection and Genotyping Technologies, Clin. Chem. 47: 164-172 (2001).
Svanvik, N. et al, Detection of PCR Products in Real-time Using Light-Up Probes, Anal. Biochem. 287:179-182 (2000).
Svanvik, N. et al, Free-Probe Fluorescence of Light-Up Probes, J. Am. Chem. Soc. 123: 803-809 (2001).
Uematru, C. et al, Multiplex polymerase chain reaction (PCR) with color-tagged module-shuffling primers for comparing gene expression levels in various cells, Nucleic Acids Res. 29: 1-6 (2001).
Walker, N.J., A Technique Whose Time Has Come, Science 296: 557-559 (2002).
Wall, S. et al, Quantitative Reverse Transcription-Polymerase Chain Reaction (RT-PCR): A Comparison of Primer-Dropping, Competitive, and Real-Time RT-PCRs, Anal. Biochem. 300: 269-273 (2002).
Wilhelm, J. et al, Influence of DNA Target Melting Behavior on Real-Time PCR Quantification Clin. Chem. 46:1738-1743 (2000).
Wittwer, C., Real-Time Multiplex PCR Assays, Methods 25: 430-442 (2001).
Vandesompele, A. et al, Elimination of Primer-Dimer Artifacts and Genomic Coamplification Using a Two-Step SYBR Green I Real-Time RT-PCR, Anal. Biochem. 303: 95-8 (2002).
Zimmermann et al, Quantitative Multiple Competitive PCR of HIV-DNA in a Single Reaction Tube, BioTechniques 21: 480-484 (1996).
Zou, Identification of New Influenza B virus Variants by Multiplex Reverse Transcription-PCR and the Heteroduplex Mobility Assay, J. Clin. Microbiol. 36: 1544-1548 (1998).

Instruction Manuals
Brilliant SYBR® Green QPCR Master Mix, Instruction Manual
Eurogentec qPCR™ Mastermix for Sybr™ Green I
SYBR® Green 1 dye for Quantitative PCR, SDS News #12
Brilliant™ SYBR® Green QPCR Master Mix
The Picofluor Method for DNA Quantification Using Hoechst 33258 Dye, Turner BioSystems
Relative Quantitation of Gene Expression, User Bulletin #2 ABI Prism 7700 Sequence Detection System, Applied Biosystems p 1-36, Dec. 11, 1997.
DNA/RNA Real-Time Quantitative PCR, Biosystems p 1-7
Sensitive, Specific Real-Time PCR Without Probes, Invitrogen LUX™ Fluorogenic Primers (2002).
Relative Quantification, Roche Applied Science, Technical Note No. LC 13/2001, p1-27.
Competitive PCR Guide, Takara Shuzo Co., Ltd.
U.S. Patents and Patent Applications
U.S. Pat. App. No. US 2002/0072112 “Thermal Cycler for Automatic Performance of the Polymerase Chain Reaction with Close Temperature Control,” Atwood et al.
U.S. Pat. No. 5,475,610 “Thermal Cycler for Automatic Performance of the Polymerase Cahin Reaction with Close Temperature Control,” Atwood et al.
U.S. Pat. App. No. 2002/0142300 “Simultaneous Screening and Identification of Sequence Alterations from Amplified Target,” Bernard et al,
U.S. Pat. No. 6,551,783 “Quantitative Analysis of Gene Expression Using PCR,” Carey et al.
U.S. Pat. No. 5,747,251 “Polymerase Chain Reaction Assays to Determine the Presence and Concentration of a Target Nucleic Acid in a Sample,” Carson et al.
U.S. Pat. No. 6,465,638 “Multiplexed PCR Assay for Detecting Disseminated Mycobacterium Avium Complex Infection,” Gorman et al.
U.S. Pat. App. No. 2003/0087397 “Multiplex Real-Time PCR,” Klein et al.
U.S. Pat. App. No. 2003/0096986 “Methods and Computer Software Products for Selecting Nucleic Acid Probes,” Mei, et al.
U.S. Pat. App. No. 2002/0058255 “PCR Reaction Mixture for Fluorescence-Based Gene Expression and Gene Mutation Analyses,” Thum et al.
U.S. Pat. App. No. 2001/0007759 “Method for Rapid Thermal Cycling of Biological Samples,” Wittwer et al.
U.S. Pat. App. No. 2002/0028452 “Method for Quantification of An Analyte,” Wittwer et al.
U.S. Pat. App. No. 2002/0058258 “Monitoring Hybridization During PCR Using SYBR Green 1,” Wittwer et al.
U.S. Pat. App. No. 2002/0123062 “Automated Analysis of Real-Time Nucleic Acid Amplification,” Wittwer.
U.S. Pat. App. No. 2003/0022177 “Single-Labeled Oligonucleotide Probes for Homogeneous Nucleic Acid Sequence Analysis,” Wittwer et al.
U.S. Pat. No. 6,174,670 “Monitoring Amplification of DNA During PCR,” Wittwer et al.
U.S. Pat. No. 6,232,079 “PCR Method for Nucleic Acid Quantification Utilizing Second or Third Order Rate Constants,” Wittwer et al.
U.S. Pat. No. 6,245,514 “Fluorescent Donor-Acceptor Pair with Low Spectral Overlap,” Wittwer et al.
U.S. Pat. No. 6,303,305 “Method for Quantification of an Analyte,” Wittwer et al.
U.S. Pat. No. 6,472,156 “Homogenous Multiplex Hybridization Analysis by Color and TM,” Wittwer et al.
U.S. Pat. No. 6,503,720 “Method for Quantification of an Analyte,” Wittwer et. U.S. Pat. No. 6,569,627 “Monitoring Hybridization During PCR Using SYBR™ Green I,” Wittwer et al.

Having generally described the present invention, the same will be better understood by reference to certain specific examples, which are set forth herein for the purpose of illustration.

### Examples

### Example I

### Cell Culture

HMC-1 mast cells were obtained from American Tissue Culture Collection (ATCC, Manassas, Va.) and were maintained in RPMI 1640 (Invitrogen, Carlsbad, Calif.) containing 10% fetal bovine serum (Invitrogen) and supplemented with 100 uM MTG (Sigma, St Louis, Mo.). Freshly fed mast cells were equally seeded into T-175 culture flasks and maintained until 70% confluent. At this time, flasks were exposed to variable amounts (1, 10, 20, 40 nM) of phorbol ester (PMA, Sigma) for 24 h.

### Example II

### RNA Extraction and Reverse Transcriptase (RT) Reaction

Total RNA from HMC-1 cells was extracted with RNeasy Mini Kit (QIAGEN) according to the manufacture's protocol and stored at −80° C. until used. 3.5 ug of total RNA were reverse-transcribed to cDNA using SuperScript First-Strand synthesis system (Invitrogen) following manufacturer's instructions.

### Example II

### Primers

Primers were designed using Primer Express v2.0 (Applied Biosystems, Foster City, Calif.) and ordered from MWG Biotech Inc (High Point, N.C.). In order to standardize real-time PCR conditions all primer sets had a calculated annealing temperature of 60° C. The set of primers used simultaneously for quantitative multiplex real-time PCR were calculated to generate amplicons with different melting temperatures (see Table 1 for details).

TABLE I

Amplicons

| | | |
|---|---|---|
| Gene | Primers 5′→3′ | Length |

| | | | | | | |
|---|---|---|---|---|---|---|
| Name | Acc No* | Forward | Reverse | Tₘ | No | (nt) |

| | | | | | | |
|---|---|---|---|---|---|---|
| Actin | NM_001101 | ACAATGAGCTGCGTGTGGCT | TCTCCTTAATGTCACGCACGA | 86.5 | II | 372 |
| | | (SEQ ID 1) | (SEQ ID 2) | | | |
| FcERIG | NM_004106 | GTTTTGGTTGAACAAGGAGCG | CCTTTCGCACTTGGATCTTCAG | 81.5 | I | 125 |
| | | (SEQ ID 3) | (SEQ ID 4) | | | |

### Example IV

### Preparation of a DNA Template by PCR

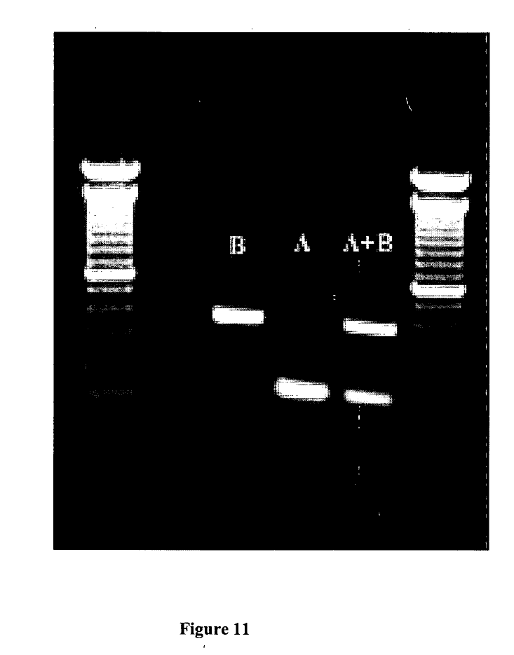

The DNA template used in some of the multiplex real-time PCR experiments was a purified PCR product. To generate the template, cDNA from the RT-reaction was amplified using the primers detailed in Table 1. The PCR reaction was run in a 25 μl volume containing 2 μl DNA template (directly from the RT-reaction), 0.4 μM each forward and reverse primers, 400 μM dNTP mix and 0.5 μl Elongase enzyme mix (Invitrogen). The PCR products were electrophoretically separated in a 1% agarose gel and the cut bands were purified using Wizard DNA Clean-Up system (Promega). The amount of each PCR product was assessed by spectrophotometry. As shown in FIG. 11, lane A represents a DNA template fragment of 125 nucleotides from the FcER1G gene, which gives rise to Amplicon I flanked by SEQ ID No.s 3 and 4. Lane B represents Amplicon II of 375 nucleotides from the Actin gene and flanked by SEQ ID Noose 1 and 2.

### Example V

### Measurement of the Tₘ of Each Amplicon

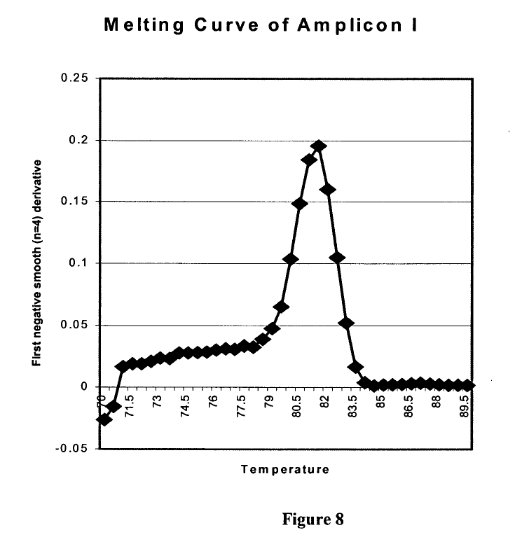

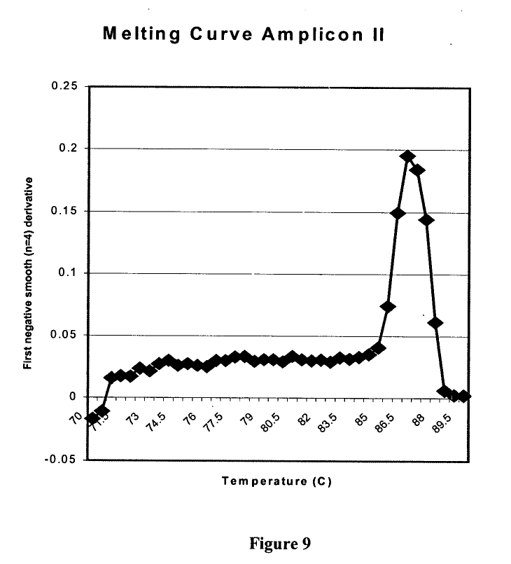

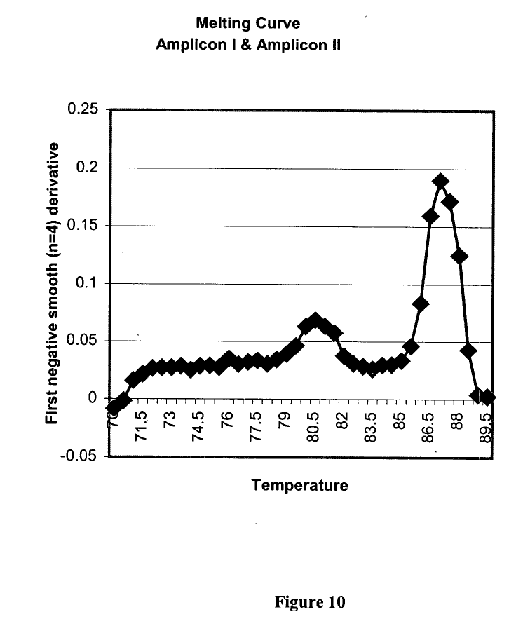

A PCR thermal cycling reaction was conducted in the presence of SYBR Green I to amplify Amplicon I alone, Amplicon II alone, and a mixture of Amplicon I and Amplicon II together in a single reaction. After the PCR reaction was completed, the fluorescence emission of SYBR Green I was read every 0.5° C. as temperature slowly rose from 70° C. to 90° C. The first negative derivative of the emission reading versus temperature was plotted and the peaks of the melting curves represented Tₘs of amplicons. FIG. 8 shows the melting curve of Amplicon I after being amplified by itself in a PCR reaction. The peak in FIG. 8 corresponds to a Tₘ of 81.5° C. FIG. 9 shows the melting curve of Amplicon II with a Tₘ of 86.5° C. FIG. 10 shows the melting curve when Amplicons I and II were amplified together in a single reaction. The temperature of the first peak corresponds to the Tₘ of Amplicon I and the second peak corresponds to that of Amplicon II.

### Example VI

### Quantitative Real-Time PCR

The quantitative real-time PCR reactions were performed in an Opticon2 Cycler (MJ Research, Waltham, Mass., USA) using SYBR Green PCR master mix (Applied BioSystems, Foster City, Calif., USA) following manufacturer's instructions. Thermocycling was performed in a final volume of 25 μl and different master mixes were prepared for single or multiplex experiments following the general protocol in Table 2. The cycling protocol was as follows: after initial denaturation of the samples at 95° C. for 2 min, 46 cycles of 95° C. for 30 s, 60° C. for 30 s, 72° C. for 35 s, 78° C. for 10 s (taking emission reading), and 84° C. for 10 s (taking emission reading) were performed. The final PCR products were visualized through a DNA gel as shown in FIG. 11.

TABLE II

| | Volume (μl) - Final concentration | |
|---|---|---|
| | One amplicon alone | Two amplicons together |
| Forward Primer | 1 − 0.4 μM | 0.5 + 0.5 − 0.4 μM |
| Reverse Primer | 1 − 0.4 μM | 0.5 + 0.5 − 0.4 μM |
| SYBR Green Mix | 12.5 − 1x | 12.5 − 1x |
| H₂O (PCR grade) | 8.5 | 8.5 |
| cDNA | 2 | 1 + 1 |

| | |
|---|---|
| Total volume | To 25 ul |

According to Example V and as shown in FIG. 10, Tₘ₁ (the Tₘ of Amplicon I) is about 81.5° C. and Tₘ₂ (the Tₘ of Amplicon II) is about 86.5° C. The measuring temperature (MT) below Tₘ₁ (or the MT pre-Tₘ₁) used in this example was 78° C., which was 3.5° C. below the Tₘ₁. The MT between Tₘ₁ and Tₘ₂ used in this example was 84° C., which was 2.5° C. above Tₘ₁ (81.5° C.) and 2.5° C. below Tₘ₂ (86.5° C.). The MT between Tₘ₁ and Tₘ₂ could also be viewed as a MT above Tₘ₁ (or a MT post-Tₘ₁) or a MT below Tₘ₂ (or a MT pre-Tₘ₂). In each cycle, the emission reading at 78° C. (a pre-Tₘ₁ emission) corresponded to the amount of Amplicons 1 and 11 in duplex. And the emission reading at 84° C. (a post-Tₘ₁ emission or a pre-Tₘ₂ emission) corresponded to the emission amount of Amplicon II in duplex. The difference between the two readings corresponded to the amount of Amplicon I in duplex.

### Example VII

### Emission Versus Cycle Curves

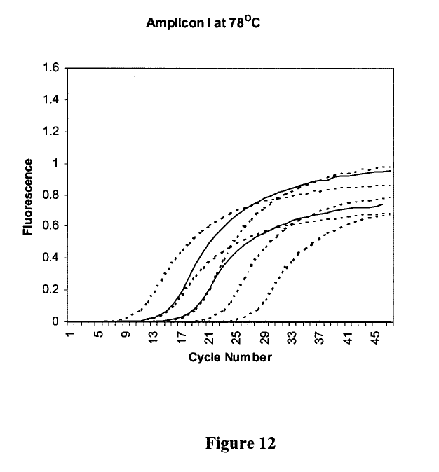

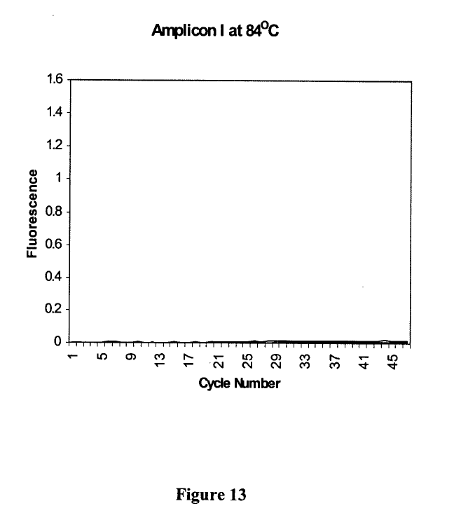

In each PCR cycle and each reaction, pre-Tₘ, emission readings taken at 78° C. and post-Tₘ₁ emission readings taken at 84° C. were recorded and plotted against the number of cycles. FIG. 12 shows standard and sample curves of Amplicon 1 at 78° C. FIG. 13 shows standard and sample curves of Amplicon 1 at 84° C. Since the Tₘ of Amplicon I is 81.5° C., Amplicon I demonstrated increasing levels of fluorescence over the cycles when the emission was measured or taken at a measuring temperature of 78° C. which was 3.5° C. lower than 81.5° C. (FIG. 12). However, no emission was detected when the emission was measured at a measuring temperature of 84° C., which was 2.5° C. higher than 81.5° C. (FIG. 13). The difference in emission is caused by the change of double stranded Amplicon I at 78° C. to single strands at 81.5° C.

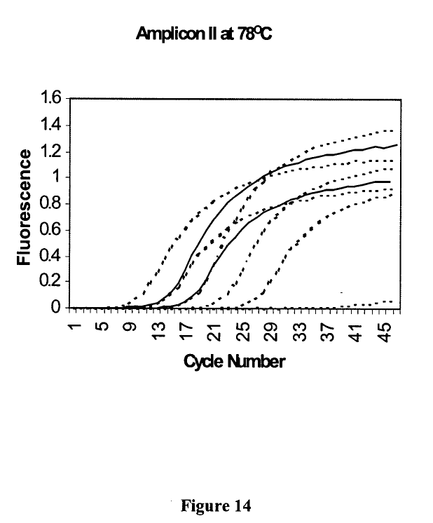

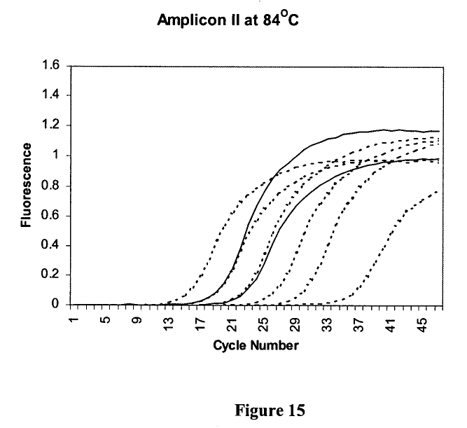

On the other hand, Amplicon II demonstrated increasing levels of fluorescence when emission was measured at both 78° C. (FIG. 14) and 84° C. (FIG. 15). Since the Tₘ of Amplicon II was 86.5° C., it was expected that Amplicon II would be in duplex at both 78° C. and 84° C.

### Example VIII

### Quantification of Amplicons Using Multiplex Protocol

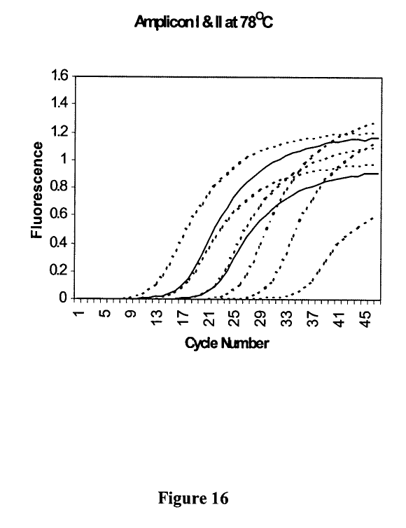

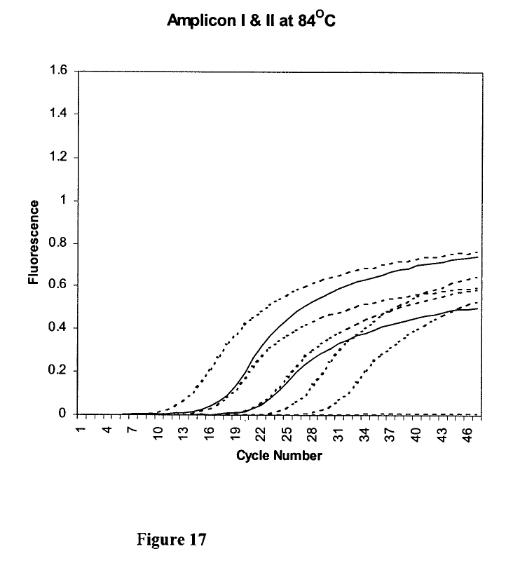

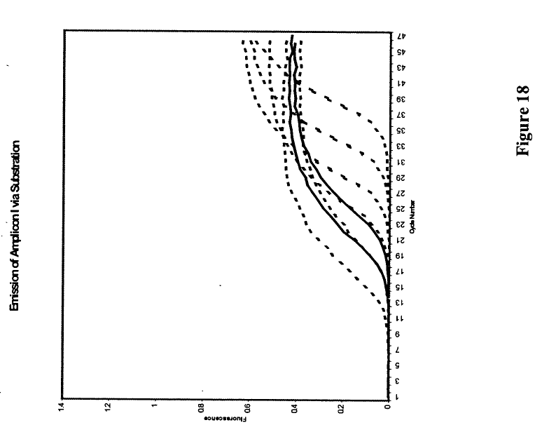

In PCR reactions containing Amplicon 1 or 11 alone, Amplicon 1 or 11 can be quantified using known software in a real-time PCR instrument based on curves as shown in FIGS. 12 and 14. In PCR reactions containing both Amplicons 1 and 11, the curves in FIG. 16 represented the emission amount of both amplicons in duplex over cycles. The curves in FIG. 17 represented the emission amount of Amplicon II in duplex over cycles. The subtraction of the emission obtained at 84° C. (as shown in FIG. 17) from the emission obtained at 78° C. (as shown in FIG. 16) from gave rise to the emission amount of Amplicon I in duplex and emission curves over cycles as shown in FIG. 18.

The subtraction of a pre-Tₘ emission from a post-Tₘ emission can be performed manually by subtracting emission data of one column (pre-Tₘ) from another (post-Tₘ). The subtraction can also be performed through a computer program or software. To expedite the quantification, software was designed to manage emission data from the multiplex real-time PCR and perform appropriate calculations. The key feature of the software was the simple subtraction of the fluorescence emission collected at a post-Tₘ measuring temperature from the fluorescence emission collected at a pre-Tₘ measuring temperature. The subtraction generated the fluorescence emission of the amplicon with the Tₘ. The software had other functions, such as manual selection of the Ct and subtraction of blanks.

The software was implemented in Visual Basic for applications (VBA) as an Addin for Microsoft Excel. The source code was organized in two main modules. One module contained all the “utility” functions such as mathematical functions, functions to generate arrays from emission data present in the Excel sheets, functions to print result data and labels, functions to handle errors or template and functions to generate charts of a certain types. The second module contained the functions to control the flow of the program. This module contained all the functions making possible the interaction with the user, such as menu selections, bar slicing, inclusion/exclusion of data in the standard curve.

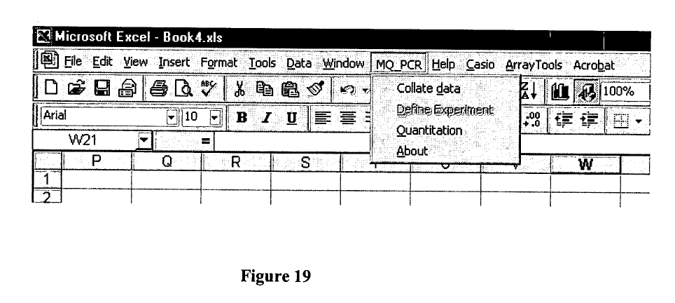

Once the Addin (called MQ_PCR or multiplex quantification real-time PCR) was installed, a menu item (called MQ_PCR) was placed in the Excel menu bar (FIG. 19). This helped not only to better organize the application but also to drive the user through the successive steps.

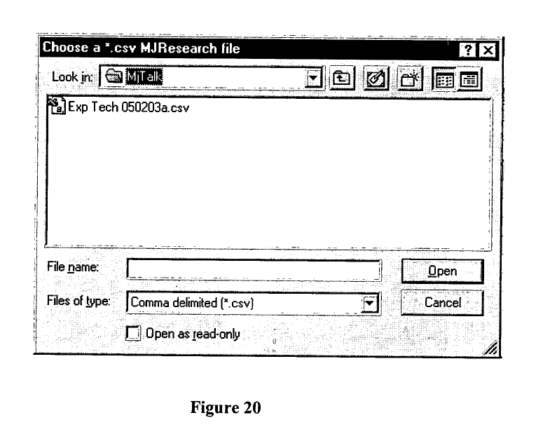

When the “Collate data” submenu was selected, a computer screen displayed a Open/Save dialog box (FIG. 20) which allowed a user to open a *.csv (comma delimited) type of file. As known in the art, csv files are the format in which the many real-time PCR instruments including Opticon2 system from MJ Research save the real-time PCR raw emission data. Alternatively, emission data can be easily converted into the cvs format. This file contained the emission data taken at each measuring temperature at every cycle for all PCR reactions. In this example, the file contained emission data for the standard curves and the samples for Amplicon I and II at temperature of 78° C. (See FIGS. 12,14 and 16) and 84° C. (See FIGS. 13, 15 and 17).

Once the file was opened, a submenu “Define Experiment” became active (FIG. 21). Selection of this submenu displayed a custom form containing three RefEdit boxes associated with three TextBoxes (FIG. 21). This allowed the user to define the cells containing the number of repeats and data for “Blanks” (cells with data for those wells lacking the cDNA but containing the rest of the reaction mix), “Standard Curve” (cells with data for the standard curves) and “Samples” (cells with data for samples). In addition the dialog box contained a TextBox to define the number of cycles run in the real-time PCR, and an “OK” and “Cancel” button.

Once the user clicked the OK button, the cells containing the data were displayed in the sheet and two additional sheets (called “baseline and results”) were generated. The data for blanks, Standard Curve and Samples were temporarily stored in three different bi-dimensional arrays. The background defined by the “Blanks” and the base line (defined as the average level of fluorescence in the first five cycles of the PCR for every sample) were subtracted from every data (however, the subtraction of blanks is optional), stored in three new arrays, and printed in the sheet named “baseline”. This step allowed the user to monitor the procedure; however, it could be executed in background.

The next step comprised the subtraction of the fluorescence emission obtained at 84° C. (FIG. 17) from the one obtained at 78° C. (FIG. 18) in each reaction. This automatically generated the “raw emission data” for the fluorescence of Amplicon I in duplex due to the lower melting temperature of 81.5° C. (FIG. 18). Since Amplicon I became single stranded at a temperature of 84° C. and generated little emission (FIG. 13), the emission amount of both amplicons obtained at 84° C. could be treated as the fluorescence emission of Amplicon II in duplex. As a result of this process, two sets of curves were obtained: one for the emission of Amplicon I (FIG. 18) and the other for emission of Amplicon II (FIGS. 17).

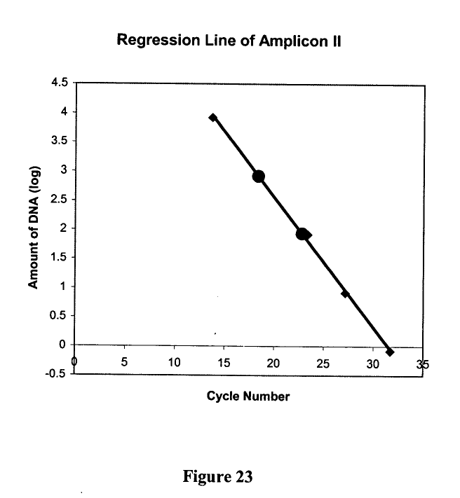

The emission curves were used to analyze Ct and regression lines. The Ct for both sets of standard curves was selected manually with the help of a scroll bar with a Ct threshold line across the standard curves (FIG. 23; this process is similar for both standard curves in FIG. 17 and FIG. 18). The scroll bar increased the cycle number (Ct) and automatically updated the regression line plot. At the same time the slope, intercept and RSquare for the regression line were shown and updated every time the user uses the sliding bar. This helped the user to select the linear part of the curves. In addition, the visualization of the RSquare value for each regression line helped to select the best fit for each regression line.

As commonly known to the art, the regression lines were used to calculate for all the samples. Based on the regression lines, in one sample, the values of Amplicons 1 and 11 in a single PCR reaction were 10.09 and 884 respectively. In a different sample, the values of Amplicons 1 and 11 were 0.98 and 78.5 respectively. As shown in Table VI, the values obtained from the methods described above were equivalent to those obtained from amplicons 1 and 11 amplified separately as well as the theoretical values, which were obtained using a spectrophotometer.

TABLE IV

| | Theoretical Value | Singleplex | Multiplex Quantitation |
|---|---|---|---|
| Amplicon II | 836 | 894 | 884 |
| | 83.6 | 68 | 78.5 |
| Amplicon I | 10.5 | 10.1 | 10.09 |
| | 1.05 | 1.01 | 0.98 |

Papers and patents listed in the disclosure are expressly incorporated by reference in their entirety. It is to be understood that the description, specific examples, and figures, while indicating preferred embodiments, are given by way of illustration and exemplification and are not intended to limit the scope of the present invention. Various changes and modifications within the present invention will become apparent to the skilled artisan from the disclosure contained herein. Therefore, the spirit and scope of the appended claims should not be limited to the description of the preferred versions contained herein.

1. A method for real-time detecting and quantifying a nucleic acid template in a PCR mixture comprising the steps of
a) thermally cycling the PCR mixture, wherein the PCR mixture comprises a thermostable polymerase, the nucleic acid template, primers to amplify at least one amplicon from the nucleic acid template, and a double stranded DNA dye, wherein the amplicon has a melting temperature of Tₘ;
b) obtaining cycle by cycle a pre-Tₘ emission at a MT below the Tₘ and a post-Tₘ emission at the a MT above the Tₘ;
c) determining cycle by cycle an emission amount of the amplicon, which is the difference between the pre-Tₘ emission and the post-Tₘ emission.

2. The method of claim 1 wherein the double stranded DNA dye is a double stranded DNA intercalating dye.

3. The method of claim 2 wherein the double stranded DNA intercalating dye is selected from the group consisting of ethidium bromide, YO-PRO-1, Hoechst 33258, SYBR Gold, and SYBR Green I.

4. The method of claim 1 wherein the double stranded DNA dye is a primer-based double stranded DNA dye.

5. The method of claims 4 wherein the primer-based double stranded DNA dye is selected from the group consisting of fluorescein, FAM, JOE, HEX, TET, Alexa Fluor 594, ROX, and TAMRA, rhodamine, BODIPY-FI.

6. The method of claim 1 wherein the MT below the Tₘ is 0.25° C. below, 0.5° C. below, 1.0° C. below, 1.5° C. below, or 2.0° C. below the Tₘ.

7. The method of claim 1 wherein the MT above the Tₘ is 0.25° C. above, 0.5° C. above, 1.0° C. above, 1.5° C. above, or 2.0° C. above the Tₘ.

8. The method of claim 1 wherein the emission amount of the amplicon is obtained through a computer program which performs a calculation of subtracting the pre-Tₘ emission from the post-Tₘ emission or the post-Tₘ emission from the pre-Tₘ emission.

9. A method for real-time detecting and quantifying a first nucleic acid template and a second nucleic acid template in a PCR mixture comprising the steps of
a) thermally cycling a PCR mixture wherein the PCR mixture comprises a thermostable polymerase, a double stranded DNA dye, the first template and the second template, primers for amplifying a first amplicon from the first template and a second amplicon from the second template, and wherein the first amplicon has a first Tₘ and the second amplicon has a second Tₘ and the first Tₘ is less than the second Tₘ;
b) obtaining cycle by cycle a first emission at a first MT between an annealing/extension temperature and the first Tₘ and a second emission at a second MT between the first Tₘ and the second Tₘ;
c) determining cycle by cycle a first emission amount of the first amplicon which is the difference between the first emission and the second emission, and a second emission amount of the second amplicon which is the second emission.

10. The method of claim 9 further comprising a step of obtaining cycle by cycle a third emission at a third MT between the second Tₘ and a total denaturing temperature, wherein the second emission amount is the difference between the second emission and the third emission.

11. The method of claim 9 wherein the double stranded DNA dye is a double stranded DNA intercalating dye.

12. The method of claim 11 wherein the double stranded DNA intercalating dye is selected from the group consisting of ethidium bromide, YO-PRO-1, Hoechst 33258, SYBR Gold, and SYBR Green I.

13. The method of claim 9 wherein the double stranded DNA dye is a primer-based double stranded DNA dye.

14. The method of claims 13 wherein the primer-based double stranded DNA dye is selected from the group consisting of fluorescein, FAM, JOE, HEX, TET, Alexa Fluor 594, ROX, and TAMRA, rhodamine, BODIPY-FI.

15. The method of claim 9 wherein the first MT is 0.25° C. below the first Tₘ, 0.5° C. below the first Tₘ, 1.0° C. below the first Tₘ, 1.5° C. below the first Tₘ, or 2.0° C. below the first Tₘ, and wherein the first MT is higher than the annealing temperature.

16. The method of claim 9 wherein the second MT is 0.25° C. below the second Tₘ, 0.5° C. below the second Tₘ, 1.0° C. below the second Tₘ, 1.5° C. below the second Tₘ, or 2.0° C. below the second Tₘ, and wherein the second MT is higher than the first Tₘ.

17. The method of claim 9 wherein the second MT is 0.25° C. above the first Tₘ, 0.5° C. above the first Tₘ, 1.0° C. above the first Tₘ, 1.5° C. above the first Tₘ, or 2.0° C. above the first Tₘ, and wherein the second MT is less than the second Tₘ.

18. The method of claim 9 wherein the second MT is the first Tₘ+0.25° C.<the second MT<the second Tₘ−0.25° C., the first Tₘ+0.5° C.<the second MT<the second Tₘ−0.5° C., the first Tₘ+1.0° C.<the second MT<the second Tₘ−1.0° C., the first Tₘ+1.5° C.<the second MT<the second Tₘ−1.5° C., or the first Tₘ+2.0° C.<the second MT<the second Tₘ−2.0° C.

19. The method of claim 10 wherein the third MT is 0.25° C. above the second Tₘ, 0.5° C. the second Tₘ, 1.0° C. above the second Tₘ, 1.5° C. above the second Tₘ, or 2.0° C. above the second Tₘ, and wherein the third MT is less than the total denaturing temperature.

20. The method of claim 9 wherein the emission amount of the first amplicon is obtained through a computer program performing a calculation of subtracting the first emission from the second emission or subtracting the second emission from the first emission.

21. A method for real-time detecting and quantifying a first nucleic acid template and a second nucleic acid template in a PCR mixture comprising the steps of:
a) thermally cycling a PCR mixture wherein the PCR mixture comprises a thermostable polymerase, a double stranded DNA dye, the first template and the second template, primers for amplifying a first amplicon from the first template and a second amplicon from the second template, and wherein the first amplicon has a first Tₘ and the second amplicon has a second Tₘ and the first Tₘ is less than the second Tₘ;
b) obtaining cycle by cycle a first pre-Tₘ emission at a MT below the first Tₘ and a first post-Tₘ emission at the a MT above the first Tₘ and a second pre-Tₘ emission at a MT below the second Tₘ and a second post-Tₘ emission at the a MT above the second Tₘ;
c) determining cycle by cycle a first emission amount of the first amplicon which is the difference between the first pre-Tₘ emission and the first post-Tₘ emission; and a second emission amount of the second amplicon which is the difference between the second pre-Tₘ emission and the second post-Tₘ emission.

22. The method of claim 21 wherein the double stranded DNA dye is a double stranded DNA intercalating dye

23. The method of claim 22 wherein the double stranded DNA intercalating dye is selected from the group consisting of ethidium bromide, YO-PRO-1, Hoechst 33258, SYBR Gold, and SYBR Green I.

24. The method of claim 21 wherein the double stranded DNA dye is a primer-based double stranded DNA dye.

25. The method of claims 24 wherein the primer-based double stranded DNA dye is selected from the group consisting of fluorescein, FAM, JOE, HEX, TET, Alexa Fluor 594, ROX, and TAMRA, rhodamine, BODIPY-FI.

26. The method of claim 21 wherein the MT below the first Tₘ and/or the second Tₘ are 0.25° C. below, 0.5° C. below, 1.0° C. below, 1.5° C. below, or 2.0° C. below.

27. The method of claim 21 wherein the MT above the first Tₘ and/or the second Tₘ are 0.25° C. above, 0.5° C. above, 1.0° C. above, 1.5° C. above, or 2.0° C. above.

28. The method of claim 21 wherein the emission amount of the amplicons is obtained through a computer program performing the calculation of subtracting the pre-Tₘ emission from the post-Tₘ emission or subtracting the post-Tₘ emission from the pre-Tₘ emission.

29. A method for real-time detecting and quantifying a total of n nucleic acid templates in a PCR mixture comprising the steps of:
a) thermally cycling a PCR mixture, wherein the PCR mixture comprises a thermostable polymerase, nucleic acid templates including n nucleic acid templates, primers for amplifying n amplicons, and a double stranded DNA dye;
b) obtaining cycle by cycle a MTₖ emission at MTₖ and MT₍ₖ₊₁₎, wherein Tₘ₍ₖ₋₁₎<MTₖ<Tₘₖ<MT₍ₖ₊₁₎<Tₘ₍ₖ₊₁₎, Tₘₖ is the Tₘ of a kth amplicon, Tₘ₍ₖ₋₁₎ is the Tₘ of a (k−1)th amplicon except that Tₘ₍ₖ₋₁₎ is an annealing and/or an extension temperature when k=1, Tₘ₍ₖ₊₁₎ is the Tₘ of a (k+1)th amplicon except that Tₘ₍ₙ₊₁₎is a total denaturing temperature when k=n, and k and n are positive integers, 1≦k≦n, and n≧2;
c) determining cycle by cycle an emission amount of the kth amplicon which is the difference between the MTₖ emission and the MT₍ₖ₊₁₎ emission.

30. The method of claim 29 wherein the double stranded DNA dye is a double stranded DNA intercalating dye.

31. The method of claim 30 wherein the double stranded DNA intercalating dye is selected from the group consisting of ethidium bromide, YO-PRO-1, Hoechst 33258, SYBR Gold, and SYBR Green I.

32. The method of claim 29 wherein the double stranded DNA dye is a primer-based double stranded DNA dye that is covalently linked to the primers.

33. The method of claims 32 wherein the primer-based double stranded DNA dye is selected from the group consisting of fluorescein, FAM, JOE, HEX, TET, Alexa Fluor 594, ROX, and TAMRA, rhodamine, BODIPY-FI.

34. The method of claim 29 wherein Tₘ₍ₖ₋₁₎+0.25° C.<MTₖ<Tₘₖ, Tₘ₍ₖ₋₁₎+0.5° C.<MTₖ<Tₘₖ, Tₘ₍ₖ₋₁₎+1.0° C.<MTₖ<Tₘₖ, Tₘ₍ₖ₋₁₎+1.5° C.<MTₖ<Tₘₖ, or Tₘ₍ₖ₋₁₎+2.0° C.<MTₖ<Tₘₖ.

35. The method of claim 29 wherein Tₘₖ+0.25° C.<MT₍ₖ₊₁₎<Tₘ₍ₖ₊₁₎, Tₘₖ+0.5° C.<MT₍ₖ₊₁₎<Tₘ₍ₖ₊₁₎, Tₘₖ+1.0° C.<MT₍ₖ₊₁₎<Tₘ₍ₖ₊₁₎, Tₘₖ+1.5° C.<MT₍ₖ₊₁₎<Tₘ₍ₖ₊₁₎, Tₘₖ+2.0° C.<MT₍ₖ₊₁₎<Tₘ₍ₖ₊₁₎.

36. The method of claim 29 wherein Tₘ₍ₖ₋₁₎<MTₖ<Tₘₖ−0.25° C., Tₘ₍ₖ₋₁₎<MTₖ<Tₘₖ−0.5° C., Tₘ₍ₖ₋₁₎<MTₖ<Tₘₖ−1.0° C., Tₘ₍ₖ₋₁₎<MTₖ<Tₘₖ−1.5° C., or Tₘ₍ₖ₋₁₎<MTₖ<Tₘₖ−2.0° C.

37. The method of claim 29 wherein Tₘₖ<MT₍ₖ₊₁₎<Tₘ₍ₖ₊₁₎−0.25° C., Tₘₖ<MT₍ₖ₊₁₎<Tₘ₍ₖ₊₁₎−0.5° C., Tₘₖ<MT₍ₖ₊₁₎<Tₘ₍ₖ₊₁₎−1.0° C., Tₘₖ<MT₍ₖ₊₁₎<Tₘ₍ₖ₊₁₎−1.5° C., Tₘₖ<MT₍ₖ₊₁₎<Tₘ₍ₖ₊₁₎−2.0° C.

38. The method of claim 29 wherein Tₘ₍ₖ₋₁₎+0.25° C.<MTₖ<Tₘₖ−0.25° C., Tₘ₍ₖ₋₁₎+0.5° C.<MTₖ<Tₘₖ−0.5° C., Tₘ₍ₖ₋₁₎+1.0° C.<MTₖ<Tₘₖ−1.0° C., Tₘ₍ₖ₋₁₎+1.5° C.<MTₖ<Tₘₖ−1.5° C. or Tₘ₍ₖ₋₁₎+2.0° C.<MTₖ<Tₘₖ−2.0° C.

39. The method of claim 29 wherein Tₘₖ+0.25° C.<MT₍ₖ₊₁₎<Tₘ₍ₖ₊₁₎−0.25° C., Tₘₖ+0.5° C.<MT₍ₖ₊₁₎<Tₘ₍ₖ₊₁₎−0.5° C., Tₘₖ+1.0° C.<MT₍ₖ₊₁₎<Tₘ₍ₖ₊₁₎−1.0° C., Tₘₖ+1.5° C.<MT₍ₖ₊₁₎<Tₘ₍ₖ₊₁₎−1.5° C., or Tₘₖ+2.0° C.<MT₍ₖ₊₁₎<Tₘ₍ₖ₊₁₎−2.0° C.

40. The method of claim 29 wherein 2≦n≦35, 2≦n≦18, 2≦n≦10, 2≦n≦7, or 2≦n≦5.

41. The method of claim 40 wherein n=2, 3, 4, or 5.

42 The method of claim 29 wherein the PCR mixture further comprises a FRET based probe.

43. The method of claim 42 wherein the FRET based probe is selected from the group consisting of a Taqman probe, a double-dye oligonucleotide probe, an Eclipse probe, a Molecular Beacon probe, a Scorpion probe, a Hybridization probe, a ResonSense probe, a Light-up probe, and a Hy-Beacon probe.

44. The method of claim 29 wherein the PCR mixture further comprises a second primer-based double stranded DNA dye that emits differently from the double stranded DNA dye.

45. The method of claim 29 wherein the emission amount of the kth amplicon is obtained through a computer program performing the subtraction of MTₖ emission from MT₍ₖ₊₁₎ emission or the subtraction of the MT₍ₖ₊₁₎ emission from MTₖ emission.

46. A method for detecting and quantifying a total of n nucleic acid templates in multiplex real-time PCR comprising the steps of:
a) thermally cycling a PCR mixture, wherein the PCR mixture comprises a thermostable polymerase, nucleic acid templates including n nucleic acid templates, primers for amplifying n amplicons, and a double stranded DNA dye;
b) obtaining cycle by cycle a pre-Tₘₖ emission of the kth amplicon at a MT between Tₘ₍ₖ₋₁₎ and Tₘₖ and a post-Tₘₖ emission of the kth amplicon at a MT between Tₘₖ and Tₘ₍ₖ₊₁₎, wherein Tₘ₍ₖ₋₁₎<Tₘₖ<Tₘ₍ₖ₊₁₎, Tₘₖ is the Tₘ of a kth amplicon, Tₘ₍ₖ₋₁₎ is the Tₘ of a (k−1)th amplicon except that Tₘ₍ₖ₋₁₎ is an annealing and/or an extension temperature when k=1, Tₘ₍ₖ₊₁₎ is the Tₘ of a (k+1)th amplicon except that Tₘ₍ₙ₊₁₎is a total denaturing temperature when k=n, and k and n are positive integers, 1≦k≦n, and n≧2;
c) determining cycle by cycle an emission amount of the kth amplicon which is the difference between the pre-Tₘₖ emission and the post-Tₘₖ emission.

47. The method of claim 46 wherein the double stranded DNA dye is a double stranded DNA intercalating dye.

48. The method of claim 47 wherein the double stranded DNA intercalating dye is selected from the group consisting of ethidium bromide, YO-PRO-1, Hoechst 33258, SYBR Gold, and SYBR Green I.

49. The method of claim 46 wherein the double stranded DNA dye is a primer-based double stranded DNA dye.

50. The method of claims 49 wherein the primer-based double stranded DNA dye is selected from the group consisting of fluorescein, FAM, JOE, HEX, TET, Alexa Fluor 594, ROX, and TAMRA, rhodamine, BODIPY-FI.

51. The method of claim 46 wherein the MT between Tₘ₍ₖ₋₁₎ and Tₘₖ is Tₘ₍ₖ₋₁₎+0.25° C.<the MT between Tₘ₍ₖ₋₁₎ and Tₘₖ<Tₘₖ, Tₘ₍ₖ₋₁₎+0.5° C.<the MT between Tₘ₍ₖ₋₁₎ and Tₘₖ<Tₘₖ, Tₘ₍ₖ₋₁₎+1.0° C.<the MT between Tₘ₍ₖ₋₁₎ and Tₘₖ<Tₘₖ, Tₘ₍ₖ₋₁₎+1.5° C.<the MT between Tₘ₍ₖ₋₁₎ and Tₘₖ<Tₘₖ, or Tₘ₍ₖ₋₁₎+2.0° C.<MTₖ<Tₘₖ.

52. The method of claim 46 wherein the MT between Tₘₖ and Tₘ₍ₖ₊₁₎is Tₘₖ+0.25° C.<the MT between Tₘₖ and Tₘ₍ₖ₊₁₎<Tₘ₍ₖ₊₁₎, Tₘₖ+0.5° C.<the MT between Tₘₖ and Tₘ₍ₖ₊₁₎<Tₘ₍ₖ₊₁₎, Tₘₖ+1.0° C.<the MT between Tₘₖ and Tₘ₍ₖ₊₁₎<Tₘ₍ₖ₊₁₎, Tₘₖ+1.5° C.<the MT between Tₘₖ and Tₘ₍ₖ₊₁₎<Tₘ₍ₖ₊₁₎, Tₘₖ+2.0° C.<the MT between Tₘₖ and Tₘ₍ₖ₊₁₎<T_{m(k+1).}

53. The method of claim 46 wherein the MT between Tₘ₍ₖ₋₁₎ and Tₘₖ is Tₘ₍ₖ₋₁₎<the MT between Tₘ₍ₖ₋₁₎ and Tₘₖ<Tₘₖ−0.25° C., Tₘ₍ₖ₋₁₎<the MT between Tₘ₍ₖ₋₁₎ and Tₘₖ<Tₘₖ−0.5° C., Tₘ₍ₖ₋₁₎<the MT between Tₘ₍ₖ₋₁₎ and Tₘₖ<Tₘₖ−1.0° C., Tₘ₍ₖ₋₁₎<the MT between Tₘ₍ₖ₋₁₎ and Tₘₖ<Tₘₖ−1.5° C., or Tₘ₍ₖ₋₁₎<the MT between Tₘ₍ₖ₋₁₎ and Tₘₖ<Tₘₖ−2.0° C.

54. The method of claim 46 wherein the MT between Tₘₖ and Tₘ₍ₖ₊₁₎ is Tₘₖ<the MT between Tₘₖ and Tₘ₍ₖ₊₁₎<Tₘ₍ₖ₊₁₎−0.25° C., Tₘₖ<the MT between Tₘₖ and Tₘ₍ₖ₊₁₎<Tₘ₍ₖ₊₁₎−0.5° C., Tₘₖ<the MT between Tₘₖ and Tₘ₍ₖ₊₁₎<Tₘ₍ₖ₊₁₎−0.0° C., Tₘₖ<the MT between Tₘₖ and Tₘ₍ₖ₊₁₎<Tₘ₍ₖ₊₁₎−1.5° C., Tₘₖ<the MT between Tₘₖ and Tₘ₍ₖ₊₁₎<Tₘ₍ₖ₊₁₎−2.0° C.

55. The method of claim 46 wherein the MT between Tₘ₍ₖ₋₁₎ and Tₘₖ is Tₘ₍ₖ₋₁₎+0.25° C.<the MT between Tₘ₍ₖ₋₁₎ and Tₘₖ<Tₘₖ−0.25° C., Tₘ₍ₖ₋₁₎+0.5° C.<the MT between Tₘ₍ₖ₋₁₎ and Tₘₖ<Tₘₖ−0.5° C., Tₘ₍ₖ₋₁₎+1.0° C.<the MT between Tₘ₍ₖ₋₁₎ and Tₘₖ<Tₘₖ−1.0° C., Tₘ₍ₖ₋₁₎+1.5° C.<the MT between Tₘ₍ₖ₋₁₎ and Tₘₖ<Tₘₖ−1.5° C. or Tₘ₍ₖ₋₁₎+2.0° C.<the MT between Tₘ₍ₖ₋₁₎ and Tₘₖ<Tₘₖ−2.0° C.

56. The method of claim 46 wherein the MT between Tₘₖ and Tₘ₍ₖ₊₁₎is Tₘₖ+0.25° C.<the MT between Tₘₖ and Tₘ₍ₖ₊₁₎<Tₘ₍ₖ₊₁₎−0.25° C., Tₘₖ+0.5° C.<the MT between Tₘₖ and Tₘ₍ₖ₊₁₎<Tₘ₍ₖ₊₁₎−0.5° C., Tₘₖ+1.0° C.<the MT between Tₘₖ and Tₘ₍ₖ₊₁₎<Tₘ₍ₖ₊₁₎−1.0° C., Tₘₖ+1.5° C.<the MT between Tₘₖ and Tₘ₍ₖ₊₁₎<Tₘ₍ₖ₊₁₎−1.5° C., or Tₘₖ+2.0° C.<the MT between Tₘₖ and Tₘ₍ₖ₊₁₎<Tₘ₍ₖ₊₁₎−2.0° C.

57. The method of claim 46 wherein 2≦n≦35, 2≦n≦18, 2≦n≦10, 2≦n≦7, or 2≦n≦5.

58 The method of claim 46 wherein the PCR mixture further comprises a FRET based probe.

59. The method of claim 46 wherein the FRET based probe is selected from the group consisting of a Taqman probe, a double-dye oligonucleotide probe, an Eclipse probe, a Molecular Beacon probe, a Scorpion probe, a Hybridization probe, a ResonSense probe, a Light-up probe, and a Hy-Beacon probe.

60. The method of claim 46 wherein the PCR mixture further comprises a second primer-based double stranded DNA dye that emits differently from the double stranded DNA dye.

61. The method of claim 46 wherein the emission amount of the kth amplicon is obtained through a computer program performing the subtraction of the pre-Tₘₖ emission from the post-Tₘₖ emission or the subtraction of the post-Tₘₖ emission from the pre-Tₘₖ emission

62. A computer software program for quantifying a real-time PCR amplicon which, when executed by a computer processor, performs the subtraction of a pre-Tₘ emission from a post-Tₘ emission or the subtraction of the post-Tₘ emission from the pre-Tₘ emission.

63. The computer software program of claim 62 wherein the emission was obtained from a double stranded DNA dye.

64. The computer software program of claim 62 wherein the double stranded DNA dye is a double stranded DNA intercalating dye.

65. The computer software program of claim 64 wherein the double stranded DNA intercalating dye is selected from the group consisting of ethidium bromide, YO-PRO-1, Hoechst 33258, SYBR Gold, and SYBR Green I.

66. The computer software program of claim 62 wherein the double stranded DNA dye is a primer-based double stranded DNA dye that is covalently linked to the primers.

67. The computer software program of claim 66 wherein the primer-based double stranded DNA dye is selected from the group consisting of fluorescein, FAM, JOE, HEX, TET, Alexa Fluor 594, ROX, and TAMRA, rhodamine, BODIPY-FI.

68. The computer software program of claim 62 wherein a pre-Tₘ emission is obtained at a MT below the Tₘ of the amplicon and a post-Tₘ emission is obtained at a MT above the Tₘ.

69. The computer software program of claim 68 wherein the MT below the Tₘ is 0.25° C. below, 0.5° C. below, 1.0° C. below, 1.5° C. below, or 2.0° C. below the Tₘ.

70. The computer software program of claim 68 wherein the MT above the Tₘ is 0.25° C. above, 0.5° C. above, 1.0° C. above, 1.5° C. above, or 2.0° C. above the Tₘ.

71. The computer software program of claim 62 which is stored and/or executed in a PCR instrument.

72. The computer software program of claim 62 which is stored and/or executed in a computer connected to a PCR instrument.

73. A computer program product comprising a computer memory having a computer software program, wherein the computer software program, when executed by a computer processor, performs the subtraction of a pre-Tₘ emission from a post-Tₘ emission or the subtraction of the post-Tₘ emission from the pre-Tₘ emission.

74. The computer program product of claim 73 wherein the emission was obtained from a double stranded DNA dye.

75. The computer program product of claim 73 wherein the double stranded DNA dye is a double stranded DNA intercalating dye.

76. The computer program product of claim 75 wherein the double stranded DNA intercalating dye is selected from the group consisting of ethidium bromide, YO-PRO-1, Hoechst 33258, SYBR Gold, and SYBR Green I.

77. The computer program product of claim 73 wherein the double stranded DNA dye is a primer-based double stranded DNA dye that is covalently linked to the primers.

78. The computer program product of claim 77 wherein the primer-based double stranded DNA dye is selected from the group consisting of fluorescein, FAM, JOE, HEX, TET, Alexa Fluor 594, ROX, and TAMRA, rhodamine, BODIPY-FI.

79. The computer program product of claim 73 wherein a pre-Tₘ emission is obtained at a MT below the Tₘ of the amplicon and a post-Tₘ emission is obtained at a MT above the Tₘ.

80. The computer program product of claim 79 wherein the MT below the Tₘ is 0.25° C. below, 0.5° C. below, 1.0° C. below, 1.5° C. below, or 2.0° C. below the Tₘ.

81. The computer program product of claim 79 wherein the MT above the Tₘ is 0.25° C. above, 0.5° C. above, 1.0° C. above, 1.5° C. above, or 2.0° C. above the Tₘ.

82. The computer program product of claim 73 which is stored and/or executed in a PCR instrument.

83. The computer program product of claim 73 which is stored and/or executed in a computer connected to a PCR instrument.

84. A PCR instrument comprising the computer program product of claim 73.
